(19) United States
(12) Patent Application Publication
(10) Pub. No.: US 2012/0219456 A1
(43) Pub. Date: Aug. 30, 2012

(54) Integrated Automatic Humidity Control And Decontamination System For Incubators And Other Laboratory Equipment

(75) Inventors: Robert Warren Childers, Trinity, FL (US); Xiang Qian Lin, Singapore (SG)

(73) Assignee: ESCO TECHNOLOGIES PTE LTD, Singapore (SG)

(21) Appl. No.: 13/505,863

(22) PCT Filed: Feb. 11, 2010
(86) PCT No.: PCT/SG10/00053

§ 371 Date: May 3, 2012

(51) Int. Cl. A61L 2/20; B01L 3/00

(52) U.S. Cl. 422/28; 422/547; 422/292

(57) ABSTRACT

The present invention relates generally to an integrated system and method for humidity control and vapor-phase decontamination of laboratory equipment with a multi-component vapor, one component of which is water vapor. The laboratory equipment can be a sealable enclosure such as an incubator, isolator, glove box, clean room, fume hood, safety cabinet, or centrifuge. The carrier gas flow is preferably in a multiple-pass, closed-loop recirculating mode. The humidity control and decontamination can be carried out automatically without user intervention for an indefinite period of time once the system has been installed, the required utilities connected and the desired settings including temperature and humidity level programmed.

The integrated humidity control and decontamination system can be retrofitted onto existing laboratory equipment.

### FIELD OF THE INVENTION

The present invention relates generally to an integrated system and method for humidity control and vapor-phase decontamination of laboratory equipment with a multi-component vapor, one component of which is water vapor. The laboratory equipment can be a sealable enclosure such as an incubator, isolator, glove box, clean room, fume hood, safety cabinet, or centrifuge. The carrier gas flow is preferably in a multiple-pass, closed-loop recirculating mode. The decontamination is of the entity as well as the exposed surfaces of the contents of the entity at the time of the decontamination.

### BACKGROUND OF THE INVENTION

Sterilization and/or decontamination of laboratory and production equipment and its contents can be as important as the sterilization of products and devices. A number of chapters in Aseptic Pharmaceutical Manufacturing II, incorporated herein by reference, discuss the use of barrier technology in the pharmaceutical industry and the need to decontaminate the interior of the barriers (sealable enclosures). The closed-loop flow-through decontamination systems disclosed in U.S. Pat. No. 5,173,258, U.S. Pat. No. 5,876,664, and U.S. Pat. No. 5,906,794 are useful for delivering sterilant vapors to sealable enclosures such as glove boxes, biological safety cabinets, the isolators used for sterility testing of pharmaceutical products, pharmaceutical form-fill-seal lines and small clean rooms. The open-loop flow-through decontamination system disclosed in U.S. Pat. No. 4,909,999, incorporated by reference herein, was intended for use with CO₂ incubators but could also be used with other sealable enclosures. More recent applications include decontamination of buildings such as post offices (U.S. Ser. No. 24/184,950) and decontamination of aircraft (U.S. Ser. No. 25/074,359).

Improvements are being made continuously in the methods and systems that are used to accomplish flow through sterilization as evidenced by the patents that have been filed in recent years. U.S. Pat. No. 5,445,792 and U.S. Pat. No. 5,508,009 disclose a method of maintaining a pre-determined percent saturation by adjusting the rate of hydrogen peroxide injection in response to a predetermined characteristic of the carrier gas. U.S. Pat. No. 5,876,664 and U.S. Pat. No. 5,906,794 disclose a method of controlling both percent saturation and sterilant vapor concentration. U.S. Pat. No. 5,872,359 discloses a real-time monitor and control system that controls both percent saturation and sterilant vapor concentration.

Sensors that can measure the concentration of the sterilant vapor, typically hydrogen peroxide, in the presence of water vapor are the subjects of a number of issued patents and pending applications including U.S. No. 56/000,142, U.S. Pat. No. 5,608,156, U.S. Pat. No. 5,847,392, U.S. Pat. No. 5,847,393, U.S. Pat. No. 6,189,368, U.S. Pat. No. 6,269,680, U.S. Pat. No. 6,517,775, U.S. Pat. No. 6,532,794, U.S. Pat. No. 6,537,491, U.S. Pat. No. 6,875,399, U.S. Ser. No. 22/168,289, U.S. Ser. No. 23/021,724, and U.S. Ser. No. 23/115,933. Methods to calibrate the sterilant vapor sensors are the subjects of a number of issued patents and pending applications including U.S. Pat. No. 6,581,435, U.S. Pat. No. 6,612,149, U.S. Pat. No. 6,742,378, U.S. Ser. No. 22/152,792, and U.S. Ser. No. 24/016,283.

U.S. Pat. No. 5,173,258 disclosed the closed loop flow-through system that was commercialized in the highly successful Steris VHP®1000 bio-decontamination system. This system preconditions the air in a sealable enclosure by re-circulating the air through an air dryer until it is at, or below, a pre-determined humidity level. If the air initially has little to no humidity no pre-conditioning is necessary. The air continues to circulate in a closed loop during the decontamination process with vaporized sterilant continuously being generated and mixed with the dehumidified air as it flows into the enclosure. As the partially degraded sterilant and air returns from the enclosure, it passes through a catalytic converter and an air dryer before being heated and combined with vaporized sterilant and returned to the enclosure. The continuous removal of partially degraded sterilant and replacement with freshly generated sterilant maximizes the concentration of the sterilant vapor within the enclosure.

The system disclosed in U.S. Pat. No. 5,173,258 requires regeneration when the desiccant capacity has been depleted. The regeneration process is described starting at line 34 in column 8. Regeneration is accomplished by blowing hot air through the desiccant bed to remove the moisture from the bed and discharge it to an outside exhaust.

The applicant evaluated a prototype hydrogen peroxide generator that used a round plastic cartridge filled with indicating Drierite® to dehumidify the air as it was drawn into the system by a blower and heater apparatus similar to that contained in a hair drier. The air flow was split after exiting the heater. A portion was split off and passed over the surface of a closed container filled with hydrogen peroxide before rejoining with the main flow of air. The closed container was the bowl in a commercially available ultrasonic nebulizer. The flowing air stream would pick up the mist generated by the nebulizer and carry it into the main flow of hot air where it was vaporized.

The applicant conducted a number of laboratory tests using the system. The water molecules were lighter than the hydrogen peroxide molecules and were found to be atomized more readily. The concentration of the hydrogen peroxide in the ultrasonic nebulizer bowl increased over time as was expected. The kill potential of the system was not constant over time because of the variation in concentration of the hydrogen peroxide vapor that was generated. The design, in spite of its simplicity and low cost, was abandoned in favor of systems that would meter a constant concentration of liquid hydrogen peroxide into a flash vaporizer at a controlled flow rate into a controlled flow rate air stream resulting in a repeatable, efficacious decontamination process.

Compressed air systems often use parallel desiccant columns to dry air. One is regenerated by bleeding a portion of the compressed air flow through it while the other dries the remaining air flow. The applicant connected two plastic desiccant cartridges obtained from W.A. Hammond in parallel using diverter valves to determine if one could be regenerated using heat while the other was being used to dry a flowing air stream. The plastic desiccant deformed from the hot air that flowed through it during the regeneration process. A metal cartridge was also tested and could take the high temperatures but would not always cool down enough to be re-used before the capacity of the other desiccant cartridge was exhausted.

The applicant also considered using pumps that would compress the air to high pressures before passing it through coalescing filters. A system was contemplated wherein the high pressure air flow would pass through a Vortec tube, splitting the air flow into a hot and a cold stream. The cool stream was used to reduce the temperature of the air stream entering the coalescing filter. The hot stream was used to re-heat the air after it exited the coalescing filer. The size, weight and cost of the air drying apparatus, when combined with the noise it generated and its power consumption, was enough for it to be abandoned.

The applicant also filled a hollow cylinder having inlet and outlet plenums on the top and bottom with deliquescent tablets. The tablets would draw the moisture from air as if flowed through the container. The pellets would dissolve and were disposed of in liquid form to the drain. When the system was shut down between uses, the pellets turned into a big clump of deliquescent that severely restricted the air flow during the next use. Thus, many of the concepts similar to those utilized in compressed air drying systems were abandoned.

U.S. Pat. No. 7,431,900 disclose a replaceable desiccant cartridge that could either be discarded after use, or regenerated after use. The cartridge is intended to be removed and regenerated elsewhere as disclosed in the patent application, or replaced with a new cartridge. This reusable cartridge that is disclosed in U.S. Pat. No. 7,431,900 solves the problems encountered by the applicant. It also differs from the re-usable desiccant cartridge that has been marketed for years by W.A. Hammond Company in that the W. A. Hammond plastic polycarbonate cartridge could not withstand the 400-450° F. desiccant regeneration temperatures. The desiccant in the W.A. Hammond desiccant cartridge has to be removed from the cartridge and regenerated, or replaced with fresh desiccant. W.A. Hammond has instructions for regeneration on their web site, included herein by reference.

The system disclosed in U.S. Pat. No. 5,906,794 also uses a desiccant that requires regeneration. A bypass of the air dryer is provided so that the air stream humidity can be controlled in a closed loop operation to a level that is higher than the output of the air dryer by controlling the amount of air that bypasses the air dryer. A continuously regenerating desiccant wheel could be used with this system if blowers were placed on each side of the air dryer. The pressure in the drying portion of the desiccant wheel could be controlled relative to the pressure in the regenerating portion of the wheel minimizing leakage from one side to the other.

Great Britain Patent GB 2308066A discloses a dehumidification method beginning with line 7 on page 13 that utilizes a refrigeration air dryer consisting of three heat exchangers. The first heat exchanger cools the air down to around 10° C. The second and third heat exchangers are in parallel. While one is cooled to below freezing and “on line”, the other is warmed and “off line”. Water is taken out of the air stream as ice by the cold heat exchanger while the “off line” heat exchanger is defrosting. The level of dehumidification is not easy to control with this system as ice is building up on the “on-line” heat exchanger during normal operation and this changes the dynamics of the airflow and the transfer of heat from the air stream.

The system that was disclosed in U.S. Pat. No. 4,909,999 was prototyped in the late 1980s with units being placed in service by Precision Scientific at The Salk Institute and at the University of Chicago. Initial skepticism changed as users vied to get access to the single “good incubator” out of the numerous incubators that were available. This system did not dry the air that was drawn in prior to introducing the vaporized hydrogen peroxide because it operated at 37° C. Sufficient hydrogen peroxide could be vaporized and introduced into the incoming air stream even if the incoming air stream was saturated with water vapor at an ambient temperature of 25° C. However, the incoming air stream could just as easily have been very dry. The unknown initial humidity variable was overcome by limiting the amount of hydrogen peroxide that was injected based upon an assumed 100% inlet air relative humidity and setting the required decontamination time based upon an assumed 0% inlet air relative humidity. The resulting decontamination cycle was effective and did not result in condensation. The system was never commercialized, presumably due to the increased incubator cost.

Patent application Ser. No. 27/274,858 discloses a novel approach to pre-conditioning the air stream before introducing hydrogen peroxide vapor. The air stream is chilled to a constant, very low temperature and humidified to near 100% at this low temperature. When the air stream is heated back up to the temperature of the enclosure to be decontaminated, the air stream is at a low, constant pre-sterilant injection relative humidity. This simplifies control and allows a maximum amount of hydrogen peroxide vapor to be introduced. This system could use a water bath and ice to pre-condition the air stream reducing its capital cost. The air can be chilled to below freezing temperatures by adding a solute to the water to depress it's freezing point.

All of the systems mentioned thus far are able to decontaminate laboratory equipment. All address, limiting or controlling the initial relative humidity of the carrier air stream. However, not very many products have been launched using the technology and those that have been introduced have not sold at a very high rate. Even though the Steris/AMSCO VHP® 1000 and other competing systems have been on the market for more than 15 years, less than 2000 units have been sold and put into operation. Advanced Sterilization Systems launched an instrument sterilizer product line in 1993, a few years after the VHP® 1000 was launched. More than 10,000 of ASPs Sterrad units have been sold.

Sanyo system described in U.S. Ser. No. 29/185,969 and assigned to Sanyo reduces the capital cost of equipment for decontaminating incubators with vaporized hydrogen peroxide by far more than an order of magnitude. With the Sanyo system, the user is responsible for ensuring that the humidification pan, located at the bottom of the incubator, is empty before starting the process. The user has to move the shelves to accommodate the sterilization module. The user is responsible for ensuring that the enclosure is otherwise free of condensation. The pre-decontamination temperature is controlled but the pre-decontamination humidity is not. It could be near zero or it could be near saturation. This variability limits the amount of hydrogen peroxide vapor that can be introduced. All of this is the responsibility of the user.

Sanyo's approach is reminiscent of the days when paraformaldehyde flakes were vaporized using an electric frying pan placed within the entity to be decontaminated. The user was responsible for controlling the process parameters such as humidity, time, temperature and the amount of sterilant that was vaporized. The lab was typically evacuated during the process in case of a gas leak except for the technician performing the decontamination. A canister type gas mask was kept close by just in case. Normal work could resume following overnight ventilation.

There is a need for a system that retains the automated operations and control of the decontamination process and which is less costly. There is also a need to integrate the two operations of dehumidifying and decontamination to overcome some of the difficulties of the prior art.

### SUMMARY OF THE INVENTION

A first object of the invention is an improved laboratory equipment having an enclosed chamber for storage of samples at a predetermined temperature and humidity level and in a sterile condition, the improvement comprising:—
a humidification system which automatically maintains the temperature, and humidity levels within the chamber at their preselected levels without user intervention for an indefinite period of time once the system has been installed, the required utilities connected and the desired settings including temperature and humidity level programmed; and
a decontamination system which automatically decontaminates the chamber without user intervention other than selecting the desired decontamination cycle, installing the sterilant cartridge and initializing a decontamination cycle.

Preferably, the humidification system draws air from the enclosed chamber, passes it in the form of small bubbles through a depth of water that is maintained at a controlled temperature to produce the desired humidity level and the humidified air is returned to the chamber.

Preferably, the depth of water for the humidification system is maintained by a float valve connected to a deionized water supply line.

Preferably, the decontamination is performed using a vaporous sterilant comprised of at least hydrogen peroxide vapor and water vapor.

Preferably, the decontamination system draws air from the enclosed chamber, passes it in the form of small bubbles through a depth of water that is maintained at a controlled temperature to pre-condition the air to the desired humidity level.

Preferably, the decontamination system discharges degraded vaporous sterilant and air in the form of small bubbles into a depth of water that condenses and captures the sterilant vapors and passes the air.

Preferably, the pre-conditioned air in the decontamination system is returned to the enclosed chamber for a time period sufficient to precondition the air within the enclosed chamber.

Preferably, the combination of pre-conditioned air at a controlled flow rate, the liquid sterilant at a controlled nebulization/vaporization rate and air in the enclosed chamber at a controlled temperature is sufficient to maintain the desired sterilant vapor concentration and desired percent saturation during the sterilization exposure phase.

More advantageously the depth of water in the decontamination system is chilled.

Preferably, the integrated automatic humidification and decontamination system for a laboratory equipment has a catalytic converter which allows the same component to be used for both the automatic humidification and for automatic decontamination.

Preferably, the integrated automatic humidification and decontamination system for a laboratory equipment has a syringe pump to pump the liquid sterilant vapor during decontamination.

Alternatively, the integrated automatic humidification and decontamination system for a laboratory equipment has a peristaltic pump that is used to supply the liquid sterilant vapor during decontamination.

Preferably, the integrated automatic humidification and decontamination system for a laboratory equipment has an air dryer containing desiccant to pre-condition the air during the decontamination stage.

More advantageously, the integrated automatic humidification and decontamination system for a laboratory equipment draws air form the enclosed chamber, passes it through an air dryer containing desiccant to pre-condition the air to the desired humidity level during the decontamination stage.

Preferably, the integrated automatic humidification and decontamination system is for a laboratory equipment, which is an incubator.

Preferably, the integrated automatic humidification and decontamination system is for a laboratory equipment, which is an isolator.

Preferably, the integrated automatic humidification and decontamination system is for a laboratory equipment, which is a fume hood.

Preferably, the integrated automatic humidification and decontamination system is for a laboratory equipment, which is a biosafety cabinet

A second object of the invention is a method to carry out decontamination using the integrated automatic humidification and decontamination system for a laboratory equipment comprising the following steps:—
drawing air from the enclosed chamber, passing the air in the form of small bubbles through a depth of water that is maintained at a controlled temperature to pre-condition the air for the decontamination step,
nebulizing a liquid sterilant into the precondition air stream,
vaporizing the nebulized liquid sterilant,
returning the sterilant vapor laden air to the enclosed chamber,
maintaining a desired sterilant vapor concentration and desired percent saturation during the sterilization exposure step by keeping the pre-conditioned air at a controlled flow rate, the liquid sterilant at a controlled vaporization rate and the air in the enclosed chamber at a controlled temperature
discharges degraded vaporous sterilant and air in the form of small bubbles into a depth of water that condenses and captures the sterilant vapors and passes the air, upon completion of the decontamination step,
returning the pre-conditioned air to the enclosed chamber for a time period sufficient to precondition the air within the enclosed chamber, and upon completion of the decontamination step,
the humidification system draws air from the enclosed chamber, passes it in the form of small bubbles through a depth of water that is maintained at a controlled temperature to produce the desired humidity level to continue operation of the laboratory equipment after the decontamination step.

A third object of the invention is an integrated humidification and decontamination module that can be retrofitted onto a laboratory equipment having an enclosed chamber for storage of samples at a predetermined temperature, humidity and CO₂ level and in a sterile condition, the said integrated humidification and decontamination system comprising:—
a humidification system which automatically maintains the temperature, and humidity levels within the enclosed chamber at their preselected levels without user intervention for an indefinite period of time once the system has been installed, the required utilities connected and the desired settings including temperature and humidity level programmed; and
a decontamination system which automatically decontaminates the enclosed chamber without user intervention other than selecting the desired decontamination cycle, installing the sterilant cartridge and initializing a decontamination cycle.

### BRIEF DESCRIPTION OF THE FIGURES

FIG. 1 is a figure taken from “Hydrogen Peroxide” by Schumb that shows the relationship between the concentration of an aqueous hydrogen peroxide solution and the vapor that forms above it during atmospheric pressure boiling.

FIG. 2 is a graph of droplet size versus pressure for a Lee pressure nozzle.

FIG. 3 is a drawing of an inexpensive ultra-sonic nebulizer module sold by TDK.

FIG. 4 is a top and front view of an incubator that embodies the system of the present invention.

FIG. 5 is a section view of the incubator of FIG. 4 schematically showing the humidification system.

FIG. 6 is a section view of the incubator of FIG. 4 schematically showing the decontamination/sterilization system.

FIG. 7 is of an incubator with a removable shelf module and a water pan for maintaining the humidity in the incubator chamber.

FIG. 8 is a section view of the incubator of FIG. 4 schematically showing an alternate embodiment of the decontamination/sterilization system.

FIG. 9 is a sketch of an embodiment of one of the shelves for use with the decontamination/sterilization system of the present invention.

FIG. 10 is a simulated cycle performed by the embodiments of the decontamination/sterilization system of the present invention shown in FIGS. 6 and 11.

FIG. 11 is an alternate embodiment of the system and method of the invention.

FIG. 12 is an embodiment of the preheater, injector and post heater portion of the system of FIGS. 6, 8 and 11.

FIG. 13 is an alternate embodiment of the preheater, injector and post heater portion of the system of FIGS. 6, 8 and 11.

FIG. 14 is an alternate embodiment of the preheater, injector and post heater portion of the systems of FIGS. 6, 8 and 11

FIG. 15 is a simulated cycle performed by the embodiment of the decontamination/sterilization system of the present invention shown in FIG. 8.

FIG. 16 is the simulated cycle of FIG. 15 performed in an environment wherein the ambient relative humidity is zero.

FIG. 17 shows an embodiment of the decontamination/sterilization system of the present invention placed on the right side of a CO₂ incubator.

FIGS. 18(a) and 18(b) are front left and front right views of the embodiment of the present invention from FIG. 17.

FIGS. 19(a) and 19(b) are right side and left rear views of the embodiment of the present invention from FIG. 17.

FIGS. 20(a), 20(b) and 20(c) are close ups of syringe pump 700 and syringe 690 from FIGS. 18(a), 18(b), 19(a) and 19(b).

FIGS. 21(a) and 21(b) are left front and right front views of an alternate embodiment of the present invention.

FIGS. 22(a) and 22(b) are left rear and right rear views of the embodiment of FIGS. 21(a) and 21(b).

### DETAILED DESCRIPTION OF THE INVENTION

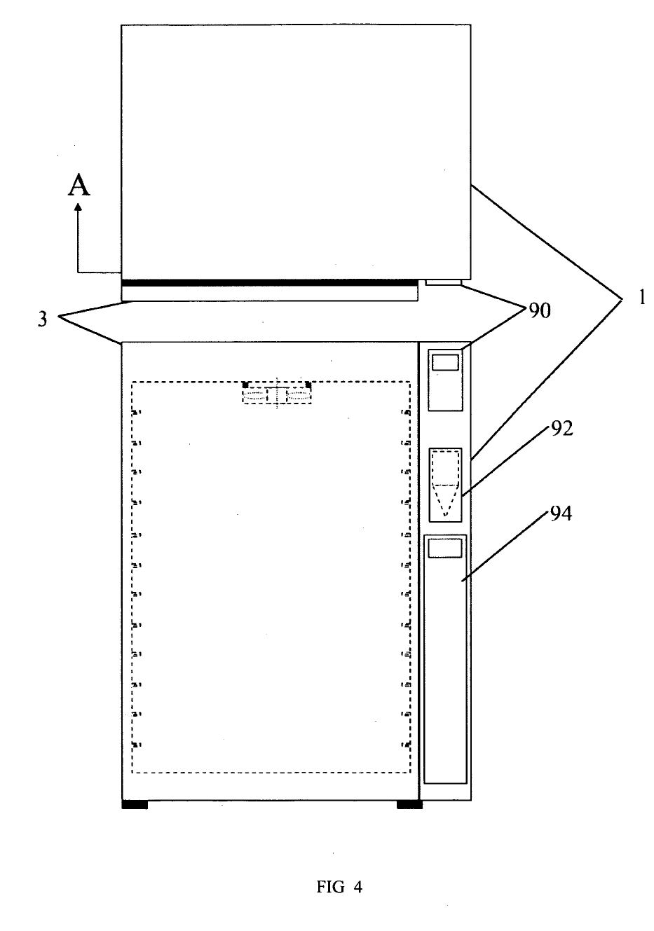

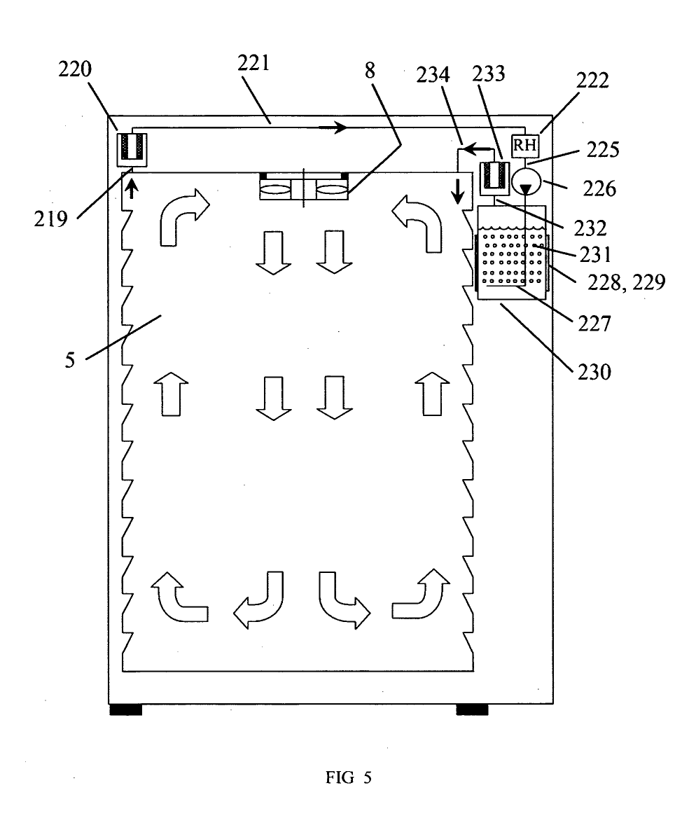

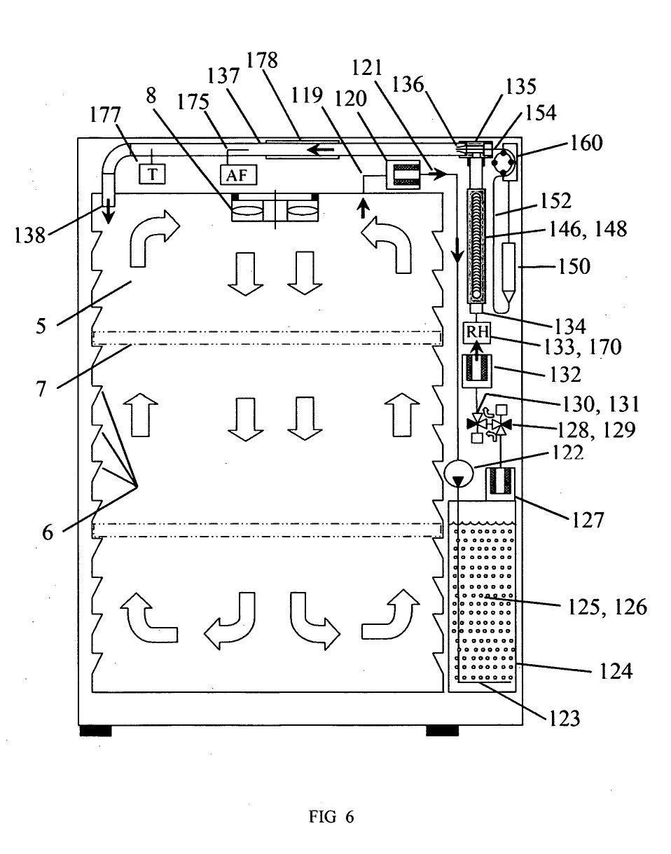

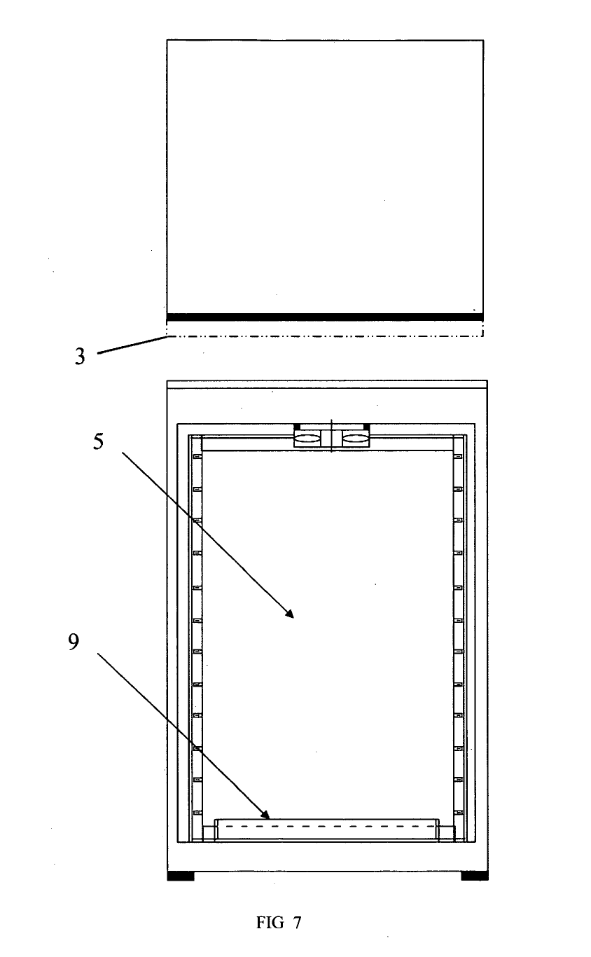

The system disclosed herein will not use an open top humidification pan 9 to maintain the humidity within the incubator chamber 5 as illustrated by FIG. 7 with door 3 removed to provide a view of the interior incubator chamber 5. The system will look more like incubator 1 shown in FIG. 4. Incubator 1 as shown in FIG. 4 will be a little wider to accommodate access portal 90 for adding water to humidification reservoir 230 shown in FIG. 5, to accommodate access portal 92 for accessing H₂O₂ supply 150 shown in FIG. 6, and to accommodate access portal 94 for accessing cold water/ice reservoir 124 shown in FIG. 6. However, the incorporation of the shelf supports into the incubator enclosure sidewalls as shown in FIGS. 4, 5, 6, 8 and 11 will minimize the increase in width due to the addition of the automated humidity control system and/or the addition of the automatic decontamination control system.

The automated humidification system, an embodiment of which is shown in FIG. 5, will add moisture when the humidity falls below that desired based upon the output of humidity sensor 222. Air will be withdrawn from incubator chamber 5 through port 219, through bacteria retentive filter 220, through conduit 221, and past humidity sensor 222 by pump 226. Pump 226 will push the air down sparger 227 into reservoir 230 that is filled with water 231 warmed by heater 228 whose temperature is monitored by temperature sensor 229. The air exiting sparger 227 “bubbles” through warm water 231 in reservoir 230 and floats to the surface where it escapes through conduit 232 and passes through bacteria retentive filter 233 and conduit 234 and is delivered to incubator chamber 5. Filter 233 may also be a coalescing filter. The air bubbles exiting reservoir 230 are saturated with water vapor producing an air stream with near 100% relative humidity. Controlling the temperature of the water in reservoir 230 indirectly allows the humidity in incubator chamber 5 to be controlled. The air stream will preferably flow in a closed loop. The air will be drawn from incubator chamber 05 by air pump 226 which is also used to “bubble” the air through heated water reservoir 230 and return it to incubator chamber 5.

Humidity sensor 222 is shown between conduit 221 and 225 in FIG. 5 but could be located elsewhere, for example, within incubator chamber 5. Placing sensor 222 as shown in FIG. 5 allows the use of a potentially lower cost humidity sensor since it does not have to withstand exposure to the H₂O₂ vapor present inside incubator chamber 5 during decontamination cycles. Sparger 227 could be fabricated from flexible tubing with many small holes through its walls or it could be fabricated from a sintered, porous material as disclosed in U.S. Ser. No. 27/274,858. Sparger 227 could also be a U-shaped piece of perforated sheet metal below which the air stream is discharged into reservoir 230.

Reservoir 230 can be connected to an appropriate supply of water (de-ionized, distilled, etc) that will allow the system to automatically maintain the water level in the reservoir. A float system (not shown) or any other known means of monitoring water level can be used to add water when the level falls below a specified level. Alternately, the user can add water through the access door whenever the system is low. The float system can cause a light/and or audible alarm to alert the user when the water level is low.

A second reservoir 124 in FIGS. 6, 8 and 11 filled with “cold” water will be used to precondition the air stream that will be used as a carrier gas during the decontamination of incubator chamber 5. The air circulation system will pass air bubbles through the cold water either reducing, or increasing, the relative humidity within the 37° C. incubator to about 40% when the incubator is set to operate at 37° C. Then, hydrogen peroxide liquid will be nebulized and swept away by the flowing stream of hot, 40% relative humidity air generating a hydrogen peroxide vapor concentration that is typically in excess of 1,400 parts per million. The preferably closed loop flow of sterilant laden air will pass into, and through, the incubator chamber before exiting and being “bubbled” through the cold water reservoir wherein the residual H₂O₂ vapor will condense and go into solution. The cold water will maintain the relative humidity of the flowing air stream. A mist, or fog, of nebulized H₂O₂ will continuously be mixed with the hot air stream that is flowing into the incubator chamber maintaining the concentration of the H₂O₂ vapor therein for the desired sterilization time.

The system controls all of the initial conditions as well as the process parameters (sterilant nebulization rate, air flow rate, air temperature, pre-injection humidity and exposure time). The liquid sterilant concentration is also controlled as it can pre-packaged and labeled with an expiration date.

The system of the present invention performs the same phases described in columns 10 and 11 of expired U.S. Ser. No. 05/173,258: Condition, Sterilization and Aeration. The system of the present invention conducts these same three phases in a closed loop manner with the carrier gas recirculating within the closed incubator system as described in U.S. Ser. No. 05/173,258. Since the sterilant is continuously being removed and replaced during the sterilization phase, the kill potential of the sterilant vapors is maximized. The process is repeatable and can be validated.

The system differs from that described in U.S. Ser. No. 05/173,258 and that described in U.S. Ser. No. 04/909,999. It does not utilize a hot surface to vaporize the liquid sterilant. It instead “flash” atomizes the sterilant into a hot, dry air stream. However, this system does not concentrate the remaining liquid sterilant as would be the case if there were a reservoir filled with liquid sterilant that is ultra-sonically nebulized.

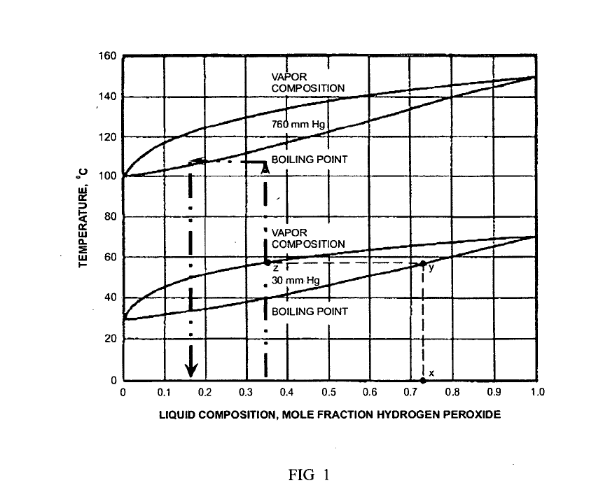

Ultrasonic nebulization from a reservoir results in the lighter water molecules being nebulized at a much higher rate than the heavier hydrogen peroxide molecules. Bubbling air through a solution of hydrogen peroxide and water produces similar results as does boiling a solution of hydrogen peroxide and water. FIG. 1 can be used to determine the hydrogen peroxide vapor concentration that can be generated by boiling a solution of a given concentration. Table 1 can be used to convert mole fraction to weight percent when using FIG. 1.

TABLE 1
Conversion Between Mole Fraction and Weight Percent

| Mole Fraction | | Mole PerCent | | Weight PerCent | |
|---|---|---|---|---|---|
| H₂O | H₂O₂ | H₂O | H₂O₂ | H₂O | H₂O₂ |
| 0 | 1 | 0.00% | 100.00% | 0.00% | 100.00% |
| 0.01 | 0.99 | 1.00% | 99.00% | 1.87% | 98.13% |
| 0.02 | 0.98 | 2.00% | 98.00% | 3.71% | 96.29% |
| 0.03 | 0.97 | 3.00% | 97.00% | 5.52% | 94.48% |
| 0.04 | 0.96 | 4.00% | 96.00% | 7.30% | 92.70% |
| 0.05 | 0.95 | 5.00% | 95.00% | 9.04% | 90.96% |
| 0.06 | 0.94 | 6.00% | 94.00% | 10.76% | 89.24% |
| 0.07 | 0.93 | 7.00% | 93.00% | 12.45% | 87.55% |
| 0.08 | 0.92 | 8.00% | 92.00% | 14.11% | 85.89% |
| 0.09 | 0.91 | 9.00% | 91.00% | 15.74% | 84.26% |
| 0.1 | 0.9 | 10.00% | 90.00% | 17.35% | 82.65% |
| 0.11 | 0.89 | 11.00% | 89.00% | 18.93% | 81.07% |
| 0.12 | 0.88 | 12.00% | 88.00% | 20.48% | 79.52% |
| 0.13 | 0.87 | 13.00% | 87.00% | 22.01% | 77.99% |
| 0.14 | 0.86 | 14.00% | 86.00% | 23.52% | 76.48% |
| 0.15 | 0.85 | 15.00% | 85.00% | 25.00% | 75.00% |
| 0.16 | 0.84 | 16.00% | 84.00% | 26.46% | 73.54% |
| 0.17 | 0.83 | 17.00% | 83.00% | 27.90% | 72.10% |
| 0.18 | 0.82 | 18.00% | 82.00% | 29.31% | 70.69% |
| 0.19 | 0.81 | 19.00% | 81.00% | 30.70% | 69.30% |
| 0.2 | 0.8 | 20.00% | 80.00% | 32.08% | 67.92% |
| 0.21 | 0.79 | 21.00% | 79.00% | 33.43% | 66.57% |
| 0.22 | 0.78 | 22.00% | 78.00% | 34.76% | 65.24% |
| 0.23 | 0.77 | 23.00% | 77.00% | 36.07% | 63.93% |

Referring to FIG. 1, taken from “Hydrogen Peroxide” by Schumb, incorporated herein by reference when a 0.22 mole fraction hydrogen peroxide solution is boiled at atmospheric pressure, a vapor with about 0.035 mole fraction is produced. If the same solution were “flash vaporized” or “flash nebulized” and then vaporized, a 0.22 mole fraction vapor would be produced. The higher vapor concentration is more desirable as it would be more effective at decontaminating the incubator or any other enclosure.

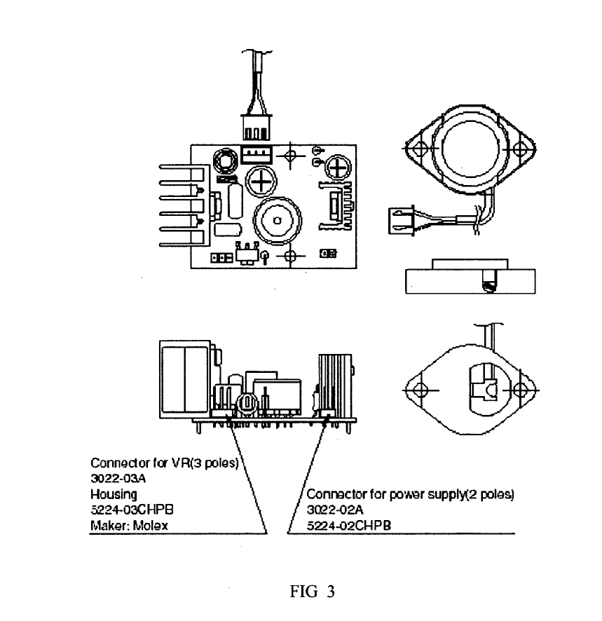

For the liquid sterilant to be ultrasonically flash nebulized, it must be delivered to the ultrasonic nebulizer surface at the rate at which it is nebulized instead of being nebulized from a reservoir filled with solution. This forces the water and hydrogen peroxide to be nebulized at the ratio defined by the liquid sterilant concentration. Inexpensive ultrasonic nebulizers such as those made by Sonaer, Inc or TDK can generate a very fine mist. FIG. 3 is a drawing of a TDK NB series ultra-sonic nebulizer.

The atomized/nebulized liquid hydrogen peroxide can be converted to vapor by heat transfer from the air stream as disclosed in U.S. Ser. No. 05/258,162. Alternately, the flowing air stream and nebulized mist can impinge on a heated surface wherein the mist is vaporized. A combination of heat transfer from the air and from droplet impingement on a surface can also be used to vaporize the mist.

The liquid sterilant can be flash atomized/nebulized by any known means before it is vaporized. Known atomizing means would include fluid pressure nozzles, pressurized air nozzles, ultrasonic atomizer nozzles, piezo-electric atomizers or the ultrasonic nebulizers discussed previously.

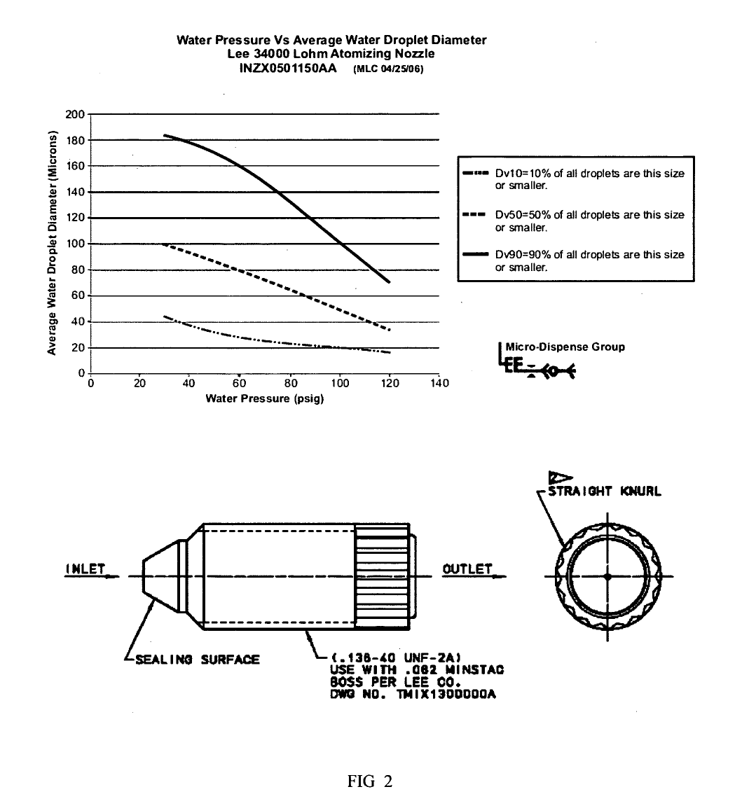

Low atomization rates can be achieved with fluid pressure nozzles by pulsing the flow to the nozzle. Pressure nozzles include the Lee Spin Jet NZSA1801600D, the Lee INZX0550050A or the Lee INZX0501150AA. FIG. 2 illustrates the droplet size produced by the Lee INZX0501150AA when H₂O is being atomized. Table 2 illustrates how pulsing the injection ON and OFF at about 35 psig injection pressure will allow a Lee nozzle to deliver over a wide range of injection rates without varying the pumping pressure.

TABLE 2
Controlling Injection Rate by Pulsing the Injector On and OFF

| | | Average Injection Rate in Grams per Minute | | | | | | | | |
|---|---|---|---|---|---|---|---|---|---|---|
| Grams | Pulse | Duty Cycle (Time Injecting/ | | | | | | | | |
| H2O2 | width | (Time Injecting + Time not Injecting) | | | | | | | | |
| Injected | (msec) | 0.50 | 0.33 | 0.25 | 0.20 | 0.17 | 0.14 | 0.13 | 0.11 | 0.10 |
| 0.0006 | 5 | 3.84 | 2.56 | 1.92 | 1.54 | 1.28 | 1.10 | 0.96 | 0.85 | 0.77 |
| 0.0016 | 10 | 4.85 | 3.23 | 2.42 | 1.94 | 1.62 | 1.38 | 1.21 | 1.08 | 0.97 |
| 0.0026 | 15 | 5.16 | 3.44 | 2.58 | 2.06 | 1.72 | 1.47 | 1.29 | 1.15 | 1.03 |
| 0.0036 | 20 | 5.37 | 3.58 | 2.69 | 2.15 | 1.79 | 1.53 | 1.34 | 1.19 | 1.07 |
| 0.0046 | 25 | 5.52 | 3.68 | 2.76 | 2.21 | 1.84 | 1.58 | 1.38 | 1.23 | 1.10 |
| 0.0055 | 30 | 5.54 | 3.70 | 2.77 | 2.22 | 1.85 | 1.58 | 1.39 | 1.23 | 1.11 |
| 0.0065 | 35 | 5.61 | 3.74 | 2.80 | 2.24 | 1.87 | 1.60 | 1.40 | 1.25 | 1.12 |
| 0.0075 | 40 | 5.65 | 3.77 | 2.82 | 2.26 | 1.88 | 1.61 | 1.41 | 1.26 | 1.13 |
| 0.0085 | 45 | 5.69 | 3.79 | 2.84 | 2.27 | 1.90 | 1.62 | 1.42 | 1.26 | 1.14 |
| 0.0095 | 50 | 5.71 | 3.80 | 2.85 | 2.28 | 1.90 | 1.63 | 1.43 | 1.27 | 1.14 |
| 0.0116 | 60 | 5.78 | 3.85 | 2.89 | 2.31 | 1.93 | 1.65 | 1.45 | 1.28 | 1.16 |
| 0.0136 | 70 | 5.81 | 3.87 | 2.91 | 2.32 | 1.94 | 1.66 | 1.45 | 1.29 | 1.16 |
| 0.0156 | 80 | 5.85 | 3.90 | 2.93 | 2.34 | 1.95 | 1.67 | 1.46 | 1.30 | 1.17 |
| 0.0175 | 90 | 5.84 | 3.89 | 2.92 | 2.33 | 1.95 | 1.67 | 1.46 | 1.30 | 1.17 |
| 0.0195 | 100 | 5.85 | 3.90 | 2.93 | 2.34 | 1.95 | 1.67 | 1.46 | 1.30 | 1.17 |

Ultra-sonic nozzles such as those manufactured by Sonics, Sonotek, Lechler and Sonicom can be used with peristaltic fluid metering pumps as they produce fine mists and they are known to not degrade the liquid sterilant during the atomization process. Minimal degradation by nebulization was confirmed by AMSCO during the development of the VHP®2000 bio-decontamination system that is disclosed in U.S. Ser. No. 05/876,664 and U.S. Ser. No. 05/906,794. A closed loop pumping arrangement was devised wherein 35% hydrogen peroxide was repeatedly nebulized into a glass beaker by a Sonics and Materials ultrasonic nozzle. The sterilant concentration had fallen by less than one percent after being continuously re-nebulized for more than 24 hours. The more expensive Sonotek ultra-sonic nozzle had been evaluated years earlier during the development of the VHP® 1000 bio-decontamination system and had also performed well.

The VHP®2000 bio-decontamination system introduced an ultra-sonically nebulized mist of hydrogen peroxide into a hot, dry air stream that passed through a continuously curving (spiral) flow path that continuously sloped downward as illustrated in figure eight of U.S. Ser. No. 05/876,664. If the hot air stream did not vaporize a droplet, an inevitable collision with the wall would vaporize it. If the rate of air flow was set too low for a given liquid injection rate and liquid started to collect on the bottom of the flow path, it would flow down the hot sloping path and be vaporized. Puddles were not allowed to form as the liquid could concentrate and could generate an explosive vapor concentration if the air flow should stop due to a power failure.

U.S. Pat. No. 4,742,667 discloses the use of compressed air to convert liquid sterilant into fine droplets that impinge on the interior surfaces of a heated tube. U.S. Ser. No. 05/068,087 and U.S. Ser. No. 04/742,667 disclose the use of compressed air nozzles to convert liquid sterilant into fine droplets that impinge on the interior surfaces of a vaporizer. U.S. Ser. No. 05/258,162 introduces a fine mist on hydrogen peroxide into an air stream that is heated sufficiently to vapor the mist.

The amount of hydrogen peroxide vapor that is generated is controlled by the flow rate from a peristaltic metering pump. A stepper motor driven pump, similar to that used in the AMSCO VHP® 1000 or a brushed DC gear motor driven pump, similar to that used on the AMSCO VHPDV30, could be used since either could produce the required flow rates. Since the nebulization/vaporization flow rate would always be the same during the decontamination process, a lookup table can be incorporated into the software to compensate for the change in liquid flow rate based upon the number of rotations and/or hours accumulated on the pump tubing. U.S. Pat. No. 4,468,219 and U.S. Pat. No. 4,346,705, both incorporated herein by reference, describe improved methods for closed loop control of a peristaltic pump.

The air flow rate is controlled by varying the speed of the air pump, blower or fan motors to produce a constant, predetermined rate of recirculating air flow through a conduit of fixed cross-section. A pitot tube air flow meter, or any other known means of measuring air flow, can provide feedback to the fan motor controller. The motor speed is increased, or decreased, to maintain the desired air flow.

The incubator temperature is controlled at its normal operating temperature which is typically 37° C. The “ON” and “OFF” time of the air stream heater, referred to as heater Duty Cycle, can be controlled based upon a temperature sensor downstream of the heater or by other know control means. A fan within the incubator will distribute the vapor and air throughout the inside of the incubator.

The method of the invention can be used to optimize the efficacy of vapor phase decontamination of an incubator in a closed loop, recirculating air flow cycle or in an open air flow cycle. Decontamination will be understood to include sterilization, disinfection and sanitization. The method of the invention is preferably used in conjunction with an automated humidity control system that eliminates the water pan 9 that is typically placed in the bottom of the chamber. A method of automated humidity control is disclosed along with the automated decontamination/sterilization method.

The sterilant vapor preferably is generated by flash nebulizing and vaporizing 30 to 35% by weight hydrogen peroxide solution to produce hydrogen peroxide vapor and water vapor. However, it could be generated by vaporizing other combinations of liquids such as peracetic acid, hydrogen peroxide and water. The carrier gas preferably is air; however, other carrier gases such as nitrogen can also be used. For the purposes of describing some of the embodiments, the carrier gas will be air and the sterilant vapor will be vapor phase hydrogen peroxide generated by flash nebulizing and vaporizing an aqueous solution of hydrogen peroxide.

In the method, a flow of carrier gas is circulated in a closed loop circuit that leads into, through and out of a sealable enclosure. The aqueous solution of hydrogen peroxide is atomized and delivered into the warm carrier gas flow wherein it is vaporized as it is being transported to the interior of the incubator. As the vapor flows through the incubator, it contacts all of the surfaces in the incubator as well as the surfaces of its contents and decontaminates them. After the carrier gas exits the sealed incubator, it passes through a cold water bath that condenses and collects the sterilant vapors. The recirculating air stream gas is warmed and returned to the incubator again laden with sterilant vapors. This circulation of the carrier gas continues for a pre-determined time that is known to affect decontamination of the incubator and its contents.

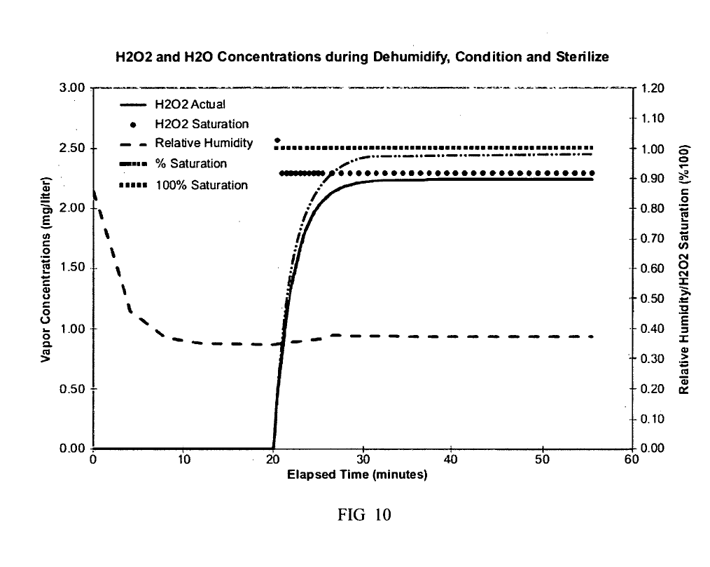

In the method of the present invention, the sterilant vapor concentration is controlled by controlling the pre-injection air stream conditions (temperature and humidity), the vaporization rate, the air flow rate and the liquid sterilant concentration. FIG. 10 is a graph of a theoretical decontamination/sterilization cycle performed by the method of the invention. This theoretical cycle was performed on a 184 liter chamber with a recirculating air flow rate of 70 liters per minute. Initially, the incubator chamber was at 100% humidity. The absolute humidity of the air stream exiting the cold water reservoir was about 15 mg/liter.

At the start of this theoretical cycle, the humidity within the 37° C. incubator chamber was reduced to its steady state value within about twenty minutes. Then, 0.55 grams per minute of 35% hydrogen peroxide was flash nebulized into the recirculating 70 liter per minute air stream, where it was vaporized and carried into the incubator chamber. 19.5 grams of 35% hydrogen peroxide solution was consumed in the 35½ minute sterilant exposure time. The relative humidity in the incubator was constant at just under 40% when H₂O₂ vapor was present. The H₂O₂ vapor reached its steady state concentration of 2.24 mg/liter within 10 minutes. The Percent saturation reached its steady state value of 98% with 10 minutes. The assumed half-life of the H₂O₂ vapor in the incubator chamber was 8.47 minutes.

Assuming that cold water reservoir 124 held 10 liters of water, the concentration of H₂O₂ in the reservoir at the end of the decontamination/sterilization cycle would be less than 0.068% (=0.35*19.5/10,000) by weight since some of the H₂O₂ would have been degraded during the vaporization/sterilization process. This would not present any hazard to the operator.

The embodiment of the system of the present invention in FIG. 6 will be used to describe the operation of the system during the hypothetical decontamination/sterilization cycle of FIG. 10.

Dehumidification Phase:

When the dehumidification phase of the decontamination cycle starts, air is drawn from incubator chamber 5 through conduit 119, bacterial retentive filter 120 and conduit 121 by air pump 122. Air pump 122 pushes the air through sparger conduit 123 that discharges the air as fine bubbles into reservoir 124 that is filled with cold water 125 and ice 126. The air bubbles float to the surface of cold water 125 and exit reservoir 124 through coalescing filter 127. The relative humidity of the air exiting reservoir 124 is around 100% at the temperature of the air when it exits reservoir 124. Assuming the air temperature is 17° C. (62.6° F.), the absolute humidity would be 15 mg/liter.

The cold air stream flows through valves 128 and 130 as well as conduits 129 and 131 and then passes through bacteria retentive filter 132 before passing by humidity sensor 170, passing through conduit 133 and into heater conduit 134 that contains heater element 146 and temperature sensor 148. Hot air exits heater conduit 134 and passes through injection tee 135, tapered orifice 136 and into warm conduit 137. Flow sensor 175 monitors the recirculating air flow rate and provides feedback to the software controlling air pump 122. Temperature sensor 177 provides feedback to the software controlling heater 178.

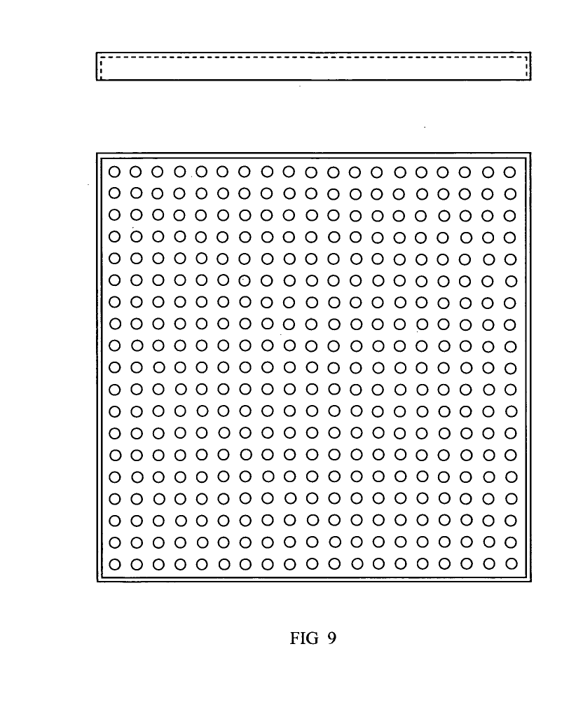

The recirculating low relative humidity air enters incubator chamber 5 through port 138. Fan 8 circulates the lower relative humidity air throughout incubator chamber 5 through perforated shelves 7 (refer to FIG. 9) so that the humidity throughout the incubator chamber 5 is nearly equal. The controlled humidity air is continuously delivered through port 138 and incubator chamber air is continuously withdrawn through conduit 119 throughout the 20 minute dehumidification phase. When the programmed dehumidification phase time has been attained, the humidity within incubator enclosure 5 is approximately equal to that which will be present during the decontamination/sterilization phase.

Condition/Decontamination Phase:

The condition/decontamination phase follows the dehumidification phase. Air continues to be drawn from incubator chamber 5 through conduit 119, bacterial retentive filter 120 and conduit 121 by air pump 122. Air pump 122 pushes the air through sparger conduit 123 that discharges the air as fine bubbles into reservoir 124 that is filled with cold water 125 and ice 126. The air bubbles float to the surface of cold water 125 and exit reservoir 124 through coalescing filter 127. The relative humidity of the air exiting reservoir 124 is around 100% at the temperature of the air when it exits reservoir 124. Assuming the air temperature is 17° C. (62.6° F.), the absolute humidity would be 15 mg/liter.

The cold air stream flows through valves 128 and 130 as well as conduits 129 and 131 and then passes through bacteria retentive filter 132 before passing by humidity sensor 170, through conduit 133 and into heater conduit 134 that contains heater element 146 and temperature sensor 148. Hot air exits heater conduit 134 and enters injection tee 135, passing through tapered orifice 136 wherein the air stream is mixed with nebulized hydrogen peroxide solution exiting from injection nozzle 154. Peristaltic pump 160 draws the liquid hydrogen peroxide solution from hydrogen peroxide supply 150 through conduit 152 and supplies it to injection nozzle 154 at a controlled rate.

The nebulized hydrogen peroxide and water mist is carried by the hot air stream down hot conduit 137 where the vaporization continues. Flow sensor 175 monitors the recirculating air flow rate and provides feedback to the software controlling the air pump. Temperature sensor 177 provides feedback to the software controlling heater 178. The sterilant vapor laden air enters incubator chamber 5 through port 138. Fan 8 circulates the sterilant laden air throughout incubator chamber 5 through perforated shelves 7 (refer to FIG. 9) so that the sterilant vapors reach all of the surfaces within the incubator chamber 5 including integrated shelf supports 6. Sterilant vapor laden air is continuously delivered through port 138 and is continuously withdrawn through conduit 119 throughout the 35½ minute exposure period maintaining the vapor concentration at about 2.24 mg/liter at a percent saturation of about 98%. When the programmed exposure time has been attained, the injection of nebulized sterilant into the heated, recirculating air stream is halted.

Aeration Phase:

The aeration phase follows the condition/decontamination phase. Diverter valve 130 is controlled to allow ambient air to enter through valve 130, flow through bacteria retentive filter 132, past humidity sensor 170, through conduit 133 and into heater conduit 134 where it is heated to 37° C. The warm air then passes through injection tee 135 and into conduit 137 where sensor 175 monitors the air flow rate and provides feedback to the circuit that controls the speed of air pump 122. The warm air then enters incubator chamber 5 through port 138. Heater 178 has a near zero duty cycle during this phase and is controlled based upon feedback from temperature sensor 177.

Fan 8 circulates the fresh, filtered air throughout incubator chamber 5 through perforated shelves 7 (refer to FIG. 9) so that the fresh air is mixed with the sterilant vapor laden air throughout the incubator chamber 5. Fresh air is continuously delivered through port 138 and air with a logarithmically decreasing sterilant vapor concentration is continuously withdrawn through conduit 119 from incubator chamber 5 throughout the aeration period.

The air that is withdrawn from incubator chamber 5 through conduit 119, bacterial retentive filter 120 and conduit 121 is pushed by air pump 122 through sparger conduit 123 that discharges the air as fine bubbles into reservoir 124 that is filled with cold water 125 and ice 126. The air bubbles float to the surface of cold water 125 and exit reservoir 124 through coalescing filter 127 and valve 128 before being discharged from the system into the room containing the incubator 1. The H₂O₂ vapor concentration in the discharged air stream is very, very low since the concentration of the H₂O₂ in the liquid in reservoir 124 is less than about 0.068%. The aeration phase ends when the predetermined aeration time has been attained.

This embodiment of the system of the present invention was able to perform a decontamination/sterilization cycle without an air dryer (desiccant), without a catalytic converter, and without connection to an outside exhaust. The power consumption is low enough for the incubator to be able to be connected to a 120V, 20 Amp electrical power outlet.

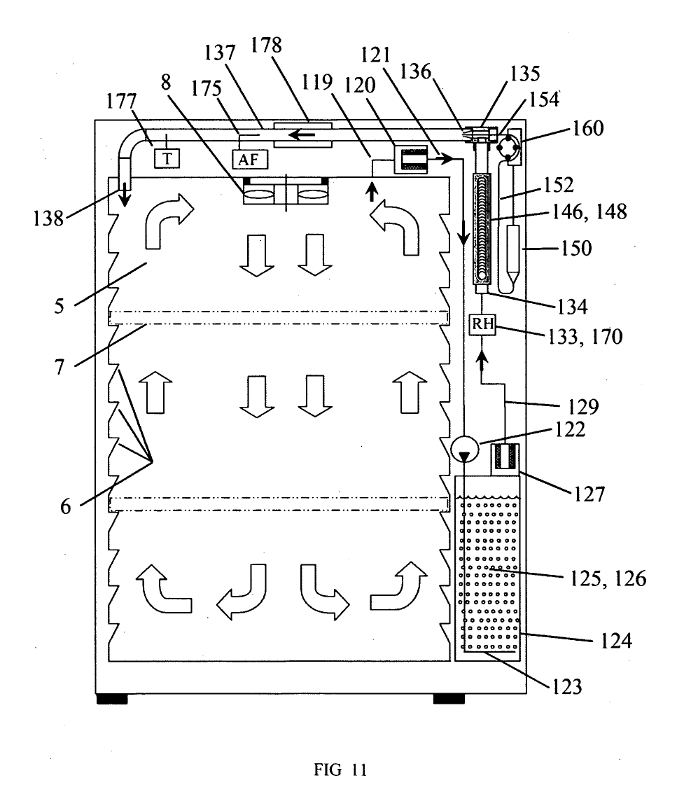

The embodiment of the system of the present invention in FIG. 11 will similarly be used to describe the operation of the system during the hypothetical decontamination/sterilization cycle of FIG. 10.

Dehumidification Phase:

When the dehumidification phase of the decontamination cycle starts, air is drawn from incubator chamber 5 through conduit 119, bacterial retentive filter 120 and conduit 121 by air pump 122. Air pump 122 pushes the air through sparger conduit 123 that discharges the air as fine bubbles into reservoir 124 that is filled with cold water 125 and ice 126. The air bubbles float to the surface of cold water 125 and exit reservoir 124 through coalescing filter 127. The relative humidity of the air exiting reservoir 124 is around 100% at the temperature of the air when it exits reservoir 124. Assuming the air temperature is 17° C. (62.6° F.), the absolute humidity would be 15 mg/liter.

The cold air stream flows through conduits 129, passes by humidity sensor 170, passes through conduit 133 and into heater conduit 134 that contains heater element 146 and temperature sensor 148. Hot air exits heater conduit 134 and passes through injection tee 135, tapered orifice 136 and into hot conduit 137. Flow sensor 175 monitors the recirculating air flow rate and provides feedback to the software controlling air pump 122. Temperature sensor 177 provides feedback to the software controlling heater 178.

The recirculating, low relative humidity air enters incubator chamber 5 through port 138. Fan 8 circulates the lower relative humidity air throughout incubator chamber 5 through perforated shelves 7 (refer to FIG. 9) so that the humidity throughout the incubator chamber 5 is nearly equal. The controlled humidity air is continuously delivered through port 138 and incubator chamber air is continuously withdrawn through conduit 119 throughout the 20 minute dehumidification phase. When the programmed dehumidification phase time has been attained, the humidity within incubator chamber 5 is approximately equal to that which will be present during the decontamination/sterilization phase.

Condition/Decontamination Phase:

The condition/decontamination phase follows the dehumidification phase. Air continues to be drawn from incubator chamber 5 through conduit 119, bacterial retentive filter 120 and conduit 121 by air pump 122. Air pump 122 pushes the air through sparger conduit 123 that discharges the air as fine bubbles into reservoir 124 that is filled with cold water 125 and ice 126. The air bubbles float to the surface of cold water 125 and exit reservoir 124 through coalescing filter 127. The relative humidity of the air exiting reservoir 124 is around 100% at the temperature of the air when it exits reservoir 124. Assuming the air temperature is 17° C. (62.6° F.), the absolute humidity would be 15 mg/liter.

The cold air stream flows through conduit 129, passes by humidity sensor 170, flows through conduit 133 and into heater conduit 134 that contains heater element 146 and temperature sensor 148. Hot air exits heater conduit 134 and enters injection tee 135, passing through tapered orifice 136 wherein the air stream is mixed with nebulized hydrogen peroxide solution exiting from injection nozzle 154. Peristaltic pump 160 draws the liquid hydrogen peroxide solution from hydrogen peroxide supply 150 through conduit 152 and supplies it to injection nozzle 154 at a controlled rate.

The nebulized hydrogen peroxide and water mist is carried by the hot air stream down hot conduit 137 where the vaporization continues. Flow sensor 175 monitors the recirculating air flow rate and provides feedback to the software controlling the air pump. Temperature sensor 177 provides feedback to the software controlling heater 178.

The sterilant vapor laden air enters incubator chamber 5 through port 138. Fan 8 circulates the sterilant laden air throughout incubator chamber 5 through perforated shelves 7 (refer to FIG. 9) so that the sterilant vapors reach all of the surfaces within the incubator chamber 5 including shelf supports 6. Sterilant vapor laden air is continuously delivered to incubator chamber 5 through port 138 and is continuously withdrawn from incubator chamber 5 through conduit 119 throughout the 35½ minute exposure period maintaining the vapor concentration at about 2.24 mg/liter at a percent saturation of about 98%. When the programmed exposure time has been attained, the injection of nebulized sterilant into the heated, recirculating air stream is halted.

Aeration Phase:

The aeration phase follows the condition/decontamination phase. Sterilant laden air is withdrawn from incubator chamber 5 through conduit 119, bacterial retentive filter 120 and conduit 121 by air pump 122. Air pump 122 pushed the air through sparger conduit 123 that discharges the air as fine bubbles into reservoir 124 that is filled with cold water 125 and ice 126. The air bubbles float to the surface of cold water 125 and exit reservoir 124 through coalescing filter 127. The H₂O₂ vapor concentration in the air exiting coalescing filter 127 is very, very low since the concentration of the H₂O₂ in the liquid in reservoir 124 is less than about 0.068%.

The air exiting coalescing filter 127 flows through conduit 129, past humidity sensor 170, through conduit 133 and into heater conduit 134 that contains heater 146 and temperature sensor 148. The air is heated to 37° C. before it passes through injection tee 135, tapered orifice 136 and into conduit 137 where sensor 175 monitors the air flow rate and provides feedback to the circuit that controls the speed of air pump 122. The warm air then enters incubator chamber 5 through port 138. Heater 178 has a near zero duty cycle during this phase and is controlled based upon feedback from temperature sensor 177.

Fan 8 circulates the filtered, nearly sterilant vapor free air throughout incubator chamber 5 through perforated shelves 7 as illustrated by FIG. 9 so that the sterilant vapor free air is mixed with the sterilant vapor laden air throughout the incubator chamber 5. Sterilant vapor free air is continuously delivered through port 138 and air with a logarithmically decreasing sterilant vapor concentration is continuously withdrawn from incubator chamber 5 through conduit 119 throughout the aeration period. The aeration phase ends when the predetermined aeration time has been attained.

This embodiment of the system of the present invention was also able to perform a decontamination/sterilization cycle without an air dryer (desiccant), without a catalytic converter, and without connection to an outside exhaust. The power consumption is low enough for the incubator 1 to be able to be connected to a 120V, 20 Amp electrical power outlet.

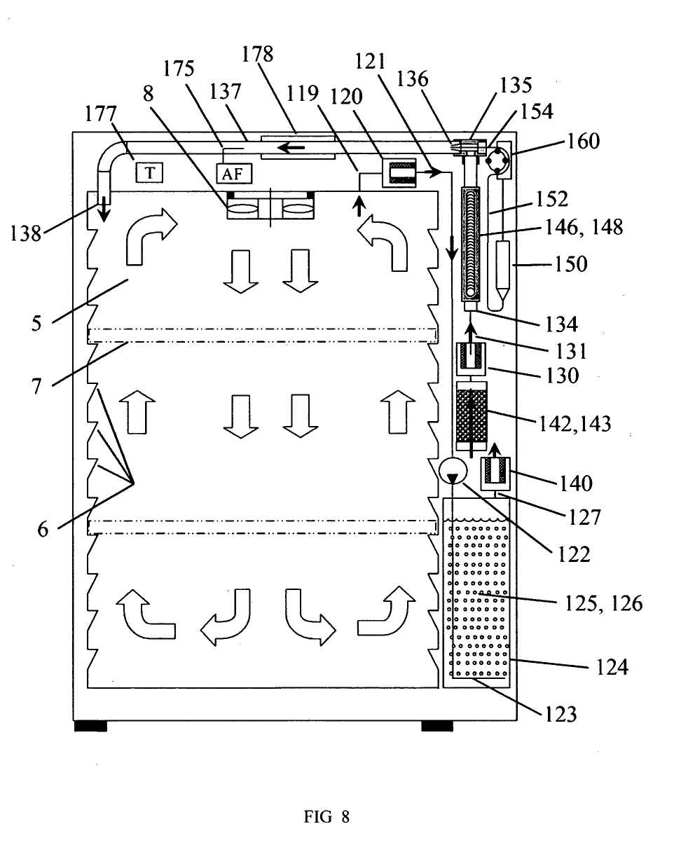

The embodiment of the system of the present invention in FIG. 8 cannot be used to describe the operation of the system during the hypothetical decontamination/sterilization cycle of FIG. 10 because it would perform a slightly different decontamination/sterilization cycle. The desiccant would lower the humidity of the recirculating air stream to about 4.6 mg/liter instead of 15 mg/liter.

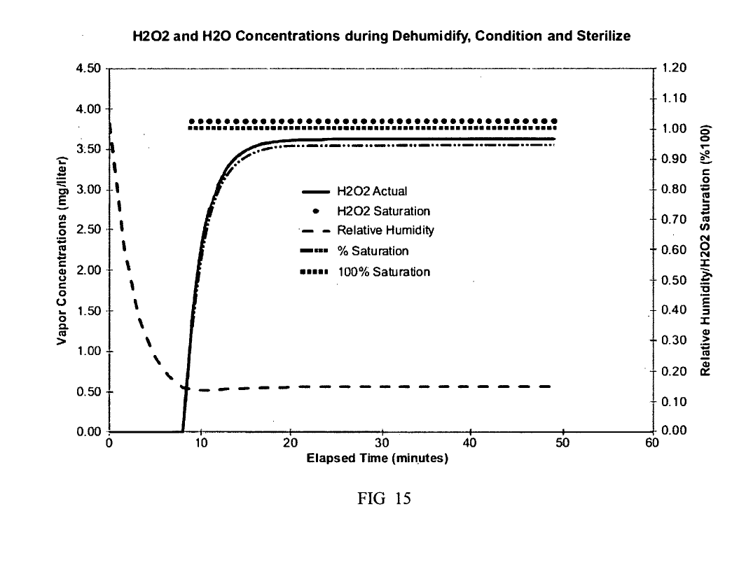

FIG. 15 is a graph of a theoretical decontamination/sterilization cycle performed by the method of the invention shown in FIG. 8. This cycle was performed on a 184 liter chamber with a recirculating air flow rate of 70 liters per minute. Initially, the incubator chamber 5 was at 100% humidity. The absolute humidity of the air stream exiting the desiccant was about 4.6 mg/liter.

At the start of this theoretical cycle, the humidity within the 37° C. incubator chamber 5 was reduced to its steady state value within eight minutes. Then, 0.89 grams per minute of 35% hydrogen peroxide was flash nebulized into the recirculating 70 liter per minute air stream, where it was vaporized and carried into the incubator chamber 5. 36.5 grams of 35% hydrogen peroxide solution was consumed in the 41 minute sterilant exposure time. The relative humidity in the incubator 1 was constant at about 15% when H₂O₂ vapor was present. The H₂O₂ vapor reached its steady state concentration of 3.63 mg/liter within about 12 minutes. The Percent saturation reached its steady state value of 94% with 10 minutes. The assumed half-life of the H₂O₂ vapor in the incubator chamber 5 was 8.47 minutes.

Assuming the cold water reservoir 124 held 10 liters of water, the concentration of H₂O₂ in the reservoir at the end of the decontamination/sterilization cycle would be less than 0.163% (=0.35*36.5/10,000) by weight since some of the H₂O₂ would have been degraded during the vaporization/sterilization process. This would not present any hazard to the operator.

The embodiment of the system of the present invention in FIG. 8 will be used to describe the operation of the system during the hypothetical decontamination/sterilization cycle of FIG. 15.

Dehumidification Phase:

Ambient air is drawn into the system of the invention through desiccant air dryer 142. The desiccant capacity has to be sufficient to absorb the moisture initially in the air within the incubator 1 plus the moisture that is in the ambient air that is drawn in during the sterilization process. Both are assumed to be at 100% RH when sizing the desiccant. The dried air then passes through conduit 143, through bacteria retentive filter 130, through conduit 131 and into conduit 134 that is warmed by heater 146. Temperature sensor 148 provides feedback to the control system that controls heater 146. The warm air exiting conduit 134 passes into injection tee 135 and through tapered orifice 136 before passing into conduit 137 that is warmed by heater 178. Temperature sensor 177 provides feedback to the software that controls heater 178. Air flow sensor 175 provides feedback to the software that controls air pump 122. The warm dry air enters incubator chamber 5 through port 138.

Fan 8 circulates the lower relative humidity air throughout incubator chamber 5 through perforated shelves 7 (refer to FIG. 9) so that the humidity throughout the incubator chamber 5 is nearly equal. The controlled humidity air is continuously delivered through port 138 and incubator chamber air is continuously withdrawn through conduit 119 throughout the 20 minute dehumidification phase. When the programmed dehumidification phase time has been attained, the humidity within incubator enclosure chamber 5 is approximately equal to that which will be present during the decontamination/sterilization phase.

Pump 122 draws the air from incubator chamber 5 through conduit 119, bacteria retentive filter 120, conduit 121 and discharges the air into reservoir 124 through sparger 123 that releases the air in the form of tiny “bubbles” that float through cold water 125 and ice 126 and surface at the top of reservoir 124 and exit into the environment through filter 140.

Condition/Decontamination Phase:

The condition/decontamination phase follows the dehumidification phase. Air continues to be drawn from incubator chamber 5 through conduit 119, bacterial retentive filter 120 and conduit 121 by air pump 122. Air pump 122 pushes the air through sparger conduit 123 that discharges the air as fine bubbles into reservoir 124 that is filled with cold water 125 and ice 126. The air bubbles float to the surface of cold water 125 and exit reservoir 124 through filter 127 to the environment.

The relative humidity of the air exiting reservoir 124 is around 100% at the temperature of the air when it exits reservoir 124. Assuming the air temperature is 17° C. (62.6° F.), the absolute humidity would be 15 mg/liter.

Ambient air is drawn into the system of the invention through desiccant air dryer 142. The dried air then passes through conduit 143, through bacteria retentive filter 130, through conduit 131 and into conduit 134 that is warmed by heater 146. Temperature sensor 148 provides feedback to the control system that controls heater 146. The warm air exiting conduit 134 passes into injection tee 135 and through tapered orifice 136 wherein the air stream is mixed with nebulized hydrogen peroxide solution exiting from injection nozzle 154. Peristaltic pump 160 draws the liquid hydrogen peroxide solution from hydrogen peroxide supply 150 through conduit 152 and supplies it to injection nozzle 154 at a controlled rate.

The nebulized hydrogen peroxide and water mist is carried by the hot air stream down hot conduit 137 where the vaporization continues. Flow sensor 175 monitors the recirculating air flow rate and provides feedback to the software controlling the air pump. Temperature sensor 177 provides feedback to the software controlling heater 178. The sterilant vapor laden air enters incubator chamber 5 through port 138.

Fan 8 circulates the sterilant laden air throughout incubator chamber 5 through perforated shelves 7 (refer to FIG. 9) so that the sterilant vapors reach all of the surfaces within the incubator chamber 5 including shelf supports 6. Sterilant vapor laden air is continuously delivered to incubator chamber 5 through port 138 and is continuously withdrawn from incubator chamber 5 through conduit 119 throughout the 41 minute exposure period maintaining the vapor concentration at about 3.63 mg/liter at a percent saturation of about 94%. When the programmed exposure time has been attained, the injection of nebulized sterilant into the heated, recirculating air stream is halted.

Aeration Phase:

The aeration phase follows the condition/decontamination phase. Sterilant laden air is withdrawn from incubator chamber 5 through conduit 119, bacterial retentive filter 120 and conduit 121 by air pump 122. Air pump 122 pushed the air through sparger conduit 123 that discharges the air as fine bubbles into reservoir 124 that is filled with cold water 125 and ice 126. The air bubbles float to the surface of cold water 125 and exit reservoir 124 through filter 127. The H₂O₂ vapor concentration in the air exiting filter 127 into the environment is very, very low since the concentration of the H₂O₂ in the liquid in reservoir 124 is less than about 0.163%.

Ambient air is drawn into the system of the invention through desiccant air dryer 142 and flows through bacteria retentive filter 132, past humidity sensor 170, through conduit 133 and into heater conduit 134 where it is heated to 37° C. The warm air then passes through injection tee 135 and tapered orifice 136 and into conduit 137 where sensor 175 monitors the air flow rate and provides feedback to the circuit that controls the speed of air pump 122. The warm air then enters incubator chamber 5 through port 138. Heater 178 has a near zero duty cycle during this phase and is controlled based upon feedback from temperature sensor 177.

Fan 8 circulates the filtered sterilant vapor free air throughout incubator chamber 5 through perforated shelves 7 (refer to FIG. 9) so that the sterilant vapor free air is mixed with the sterilant vapor laden air within the incubator chamber 5. Sterilant vapor free air is continuously delivered through port 138 and air with a logarithmically decreasing sterilant vapor concentration is continuously withdrawn from incubator chamber 5 through conduit 119 throughout the aeration period. The aeration phase ends when the predetermined aeration time has been attained.

The embodiment of FIG. 8 of the system of the present invention was able to perform a decontamination/sterilization cycle without a catalytic converter 615, and without connection to an outside exhaust. The power consumption is low enough for the incubator 1 to be able to be connected to a 120V, 20 Amp electrical power outlet.

The embodiment of FIG. 8 was able to maintain a higher H₂O₂ vapor concentration throughout the exposure phase because the pre-injection humidity of the recirculating air stream was reduced to about 4.6 mg/liter by the desiccant air dryer 142 instead of to 15 mg/liter by the cold water reservoir. This allowed a higher injection rate without condensation forming.

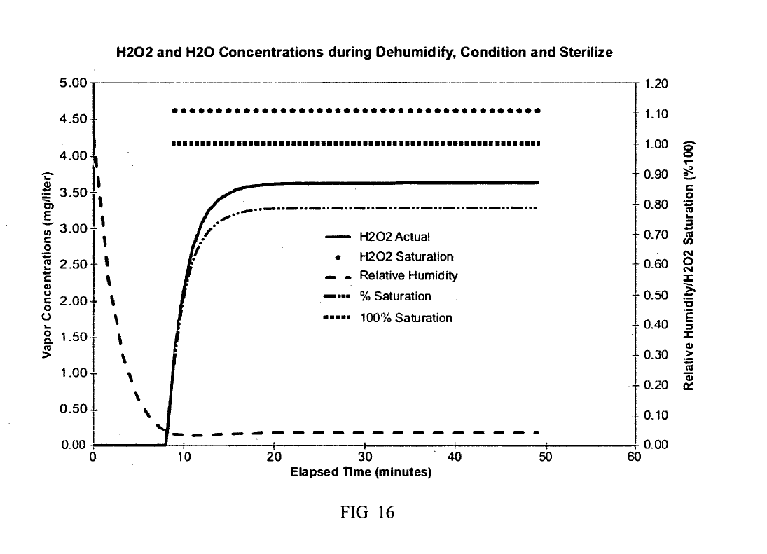

However, this embodiment of FIG. 8 is not able to raise the pre-injection humidity when the ambient humidity is lower than 4.6 mg/liter as is the case in much of the world at one time or another. The Percent saturation would be less than 94% when the environmental humidity is less than 4.6 mg/liter. The worst case Percent saturation would occur when the environmental humidity is at about 0 mg/liter. FIG. 16 is illustrative of a decontamination/sterilization cycle performed with the embodiment of FIG. 8 when the environmental humidity is at zero. The Percent saturation falls to 79% even though the H₂O₂ vapor concentration is unchanged at 3.63 mg/liter. The reduction in sterilization efficacy associated with the lower Percent saturation approximately offsets the gain in sterilization efficacy made by the increased H₂O₂ vapor concentration so that the decontamination/sterilization cycles of FIGS. 10 and 15 are about equal in terms of effectiveness when performed in a dry climate, or during a dry season.

The addition of a relative humidity sensor between air dryer 142 and heated conduit 134 would allow the embodiment of FIG. 8 to maximize both the H₂O₂ vapor concentration and the Percent saturation. The sterilant injection rate could be increased by the software controlling the system when the humidity was less than 4.6 mg/liter.

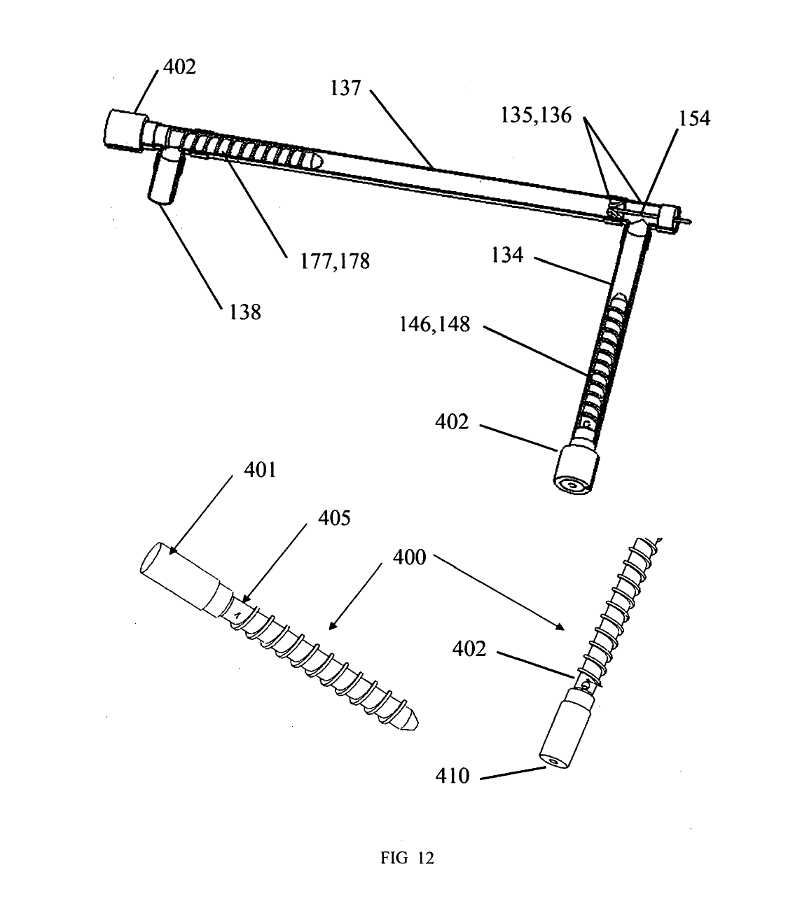

FIG. 12 is an embodiment of heated conduit 134, injection tee 154 and heated conduit 137 that could be used in the embodiments of the present invention disclosed in FIG. 6, 8 or 11. Both heaters 146 and 178 are comprised of an auger shaped spiral section 400 that is attached to a cylindrical section 401. There is a short gap 405 in the spiral feature near the cylindrical section leaving an annular volume around the auger shaft. The auger shaft of heater 146 is hollow so that air flow can pass through the center 410 of larger diameter cylindrical section, through radial holes and into the spiral flow path formed by the auger section and the interior of conduit 134. Air flows in the reverse direction down the spiral pathway formed by auger shaped heater 178 and conduit 137. When the air flow reaches the short gap 405 at the end of the auger spiral, it enters the annular volume between the auger shaft and conduit 137 that allows it to pass out through conduit port 138.

Injection tee 135 connects conduits 134 and 137 together and provides a mount for injector 154 that allows the liquid sterilant to be introduce in the flowing air stream at the smallest diameter of tapered orifice 136. The hot, flowing air stream will vaporize much of the mist that is generated by injector 154. Any of the mist that is not vaporized will vaporize as it passes down the spiral pathway of heater 178. Any entrained liquid will impact the hot spirals of auger shaped pathway 400 repeatedly until it is vaporized.

A MICA band heater element 402 can be clamped around the cylindrical section 401 of auger shaped heaters 146 and 178. Temperature sensors 148 and 177 can monitor the auger shaft temperature, the exiting air temperature or both and provide feedback to the software that controls heaters 146 and 178.

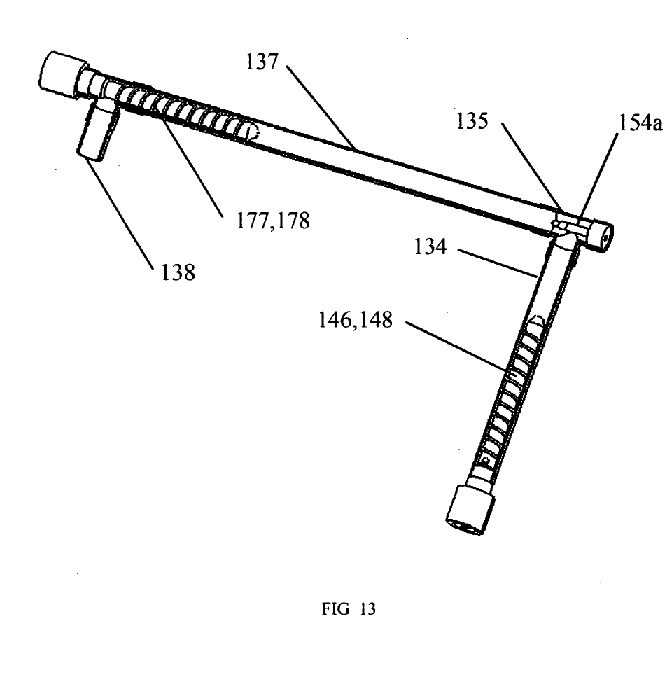

FIG. 13 is a variant of the embodiment of heated conduit 134, injection tee 135 and heated conduit 137 wherein tapered orifice 136 and injector 154 are replaced by an injector nozzle 154a such as the Lee Spin Jet that generates a cone shaped mist that is discharged into the flowing air stream on the outlet side of injection tee 154. The functionality of the embodiment of FIG. 13 is otherwise the same as that of FIG. 12.

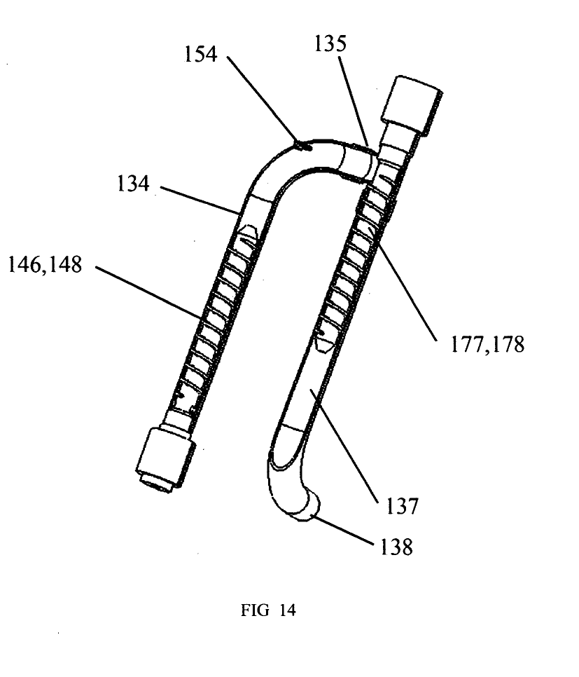

FIG. 14 is a re-arrangement of the components disclosed in FIG. 13 that is more suited to a side mount installation on incubator chamber 5. Port 138 is located near the bottom of incubator chamber 5. The spiral auger shaped section of heater 178 accommodates a top to bottom air flow.

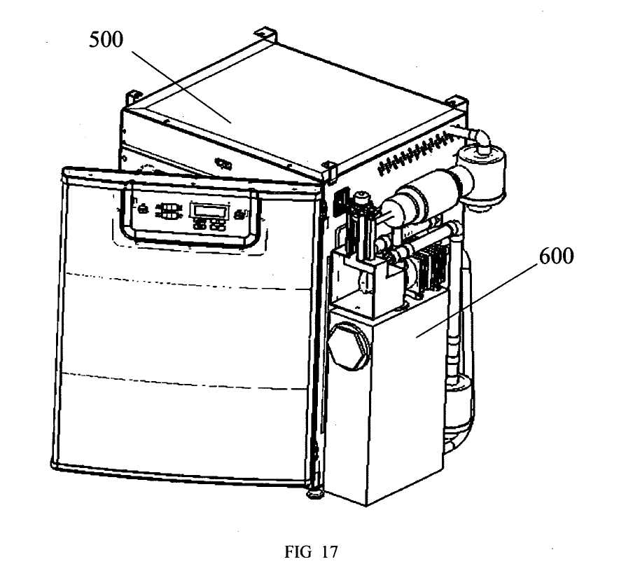

FIG. 17 shows an embodiment of the present invention 608 placed alongside a CO₂ incubator 500. If the right sidewall of the incubator were extended a few inches, the decontamination/humidity control module 600 would fit inside the incubator enclosure.

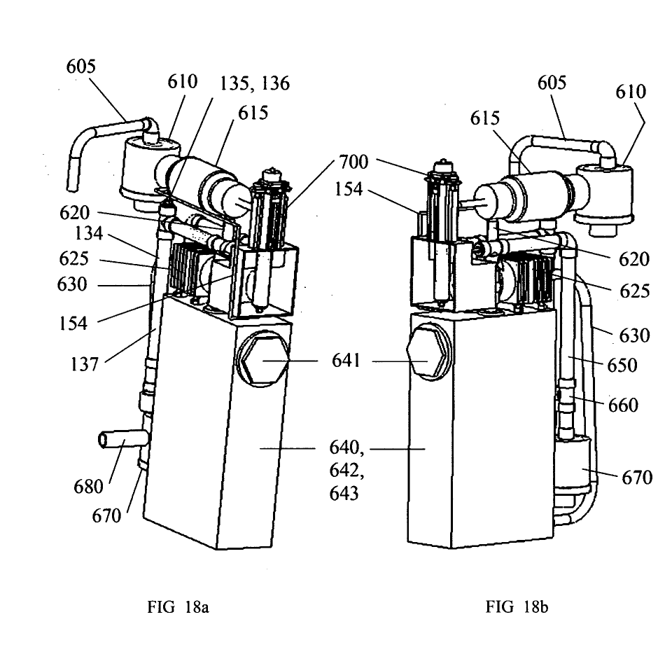

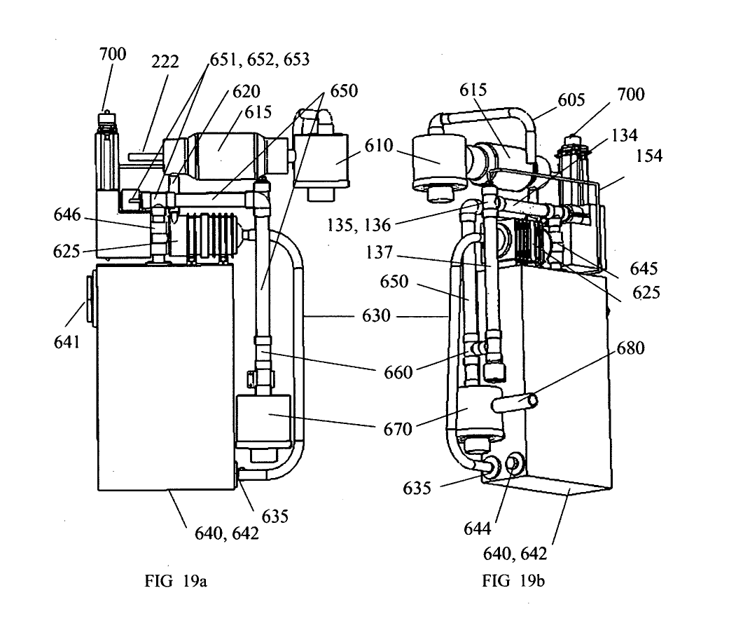

FIGS. 18(a) and 18(b) are front left and front right views of decontamination/humidity control module 600. FIGS. 19(a) and 19(b) are right side and left read view of decontamination/humidity control module 600. Referring now to FIGS. 18(a), 18(b), 19(a) and 19(b), air is withdrawn from the incubator chamber 5 through conduit 605. The air passes through HEPA filter 610, catalytic converter 615, and conduit 620 as it is drawn in by air pump 625. Air pump 625 discharges the air through conduit 630 and sparger 635 into reservoir 640 that is filled with water 642. Heater 644 is used to warm water 642 in reservoir 640 when module 600 is operating in the humidity control mode. Ice 643 is added to reservoir 640 through access port 641 when module 600 operates in the decontamination mode.

Sparger 635 extends into reservoir 640. The air that enters sparger 635 exits into water 642 in reservoir 640 as microbubbles with a high surface to volume ratio. When the microbubbles exit water 642 and enter the air filled head space at the top of reservoir 640 the air bubbles have a relative humidity of near 100% based upon the temperature of water 642 in reservoir 640. If water 642 is hot, the absolute humidity level of the air bubble is high. If water 642 is cold, the absolute humidity level of the air bubble is low. The air bubbles combine in the air space above water 642 in reservoir 640.

The air exits at the top of reservoir 640 through either valve 645 or valve 646. If valve 645 is open, valve 646 will be closed. Conversely, if valve 646 is open, valve 645 will be closed. Open valve 645 allows the air exiting reservoir 640 to pass into a heated conduit 134, injection tee 154 and heated conduit 137 as disclosed in both FIGS. 12 and 13. Open valve 646 allows the air exiting reservoir 640 to pass into and through conduit 650. Conduit 650 may optionally begin with tee 651 that will allow temperature probe 652 and/or humidity probe 653 to monitor the air exiting reservoir 640.

Conduits 137 and 650 merge at tee 660. Thus, the flow of air during humidification as well as during decontamination will pass through tee 660, into and through HEPA filter 670, and through conduit 680 as it is returned to the incubator chamber 5. During decontamination, catalytic converter 615 breaks the H₂O₂ vapor into H₂O and O₂ before it passes humidity sensor 222.

The humidification and decontamination circuits were able to be combined by the addition of catalytic converter 615. The addition of catalytic converter 615 allowed a number of components to serve a “double function”. Air pump 625 performs the functions of air pump 222 from FIG. 5 and air pump 122 from FIG. 6. HEPA filter 610 performs the functions of filter 220 from FIG. 5 and filter 120 from FIG. 6. HEPA filter 670 performs the same function as HEPA filter 233 from FIG. 5 and HEPA filter 132 from FIG. 6. Reservoir 640 performs the same function as reservoir 230 from FIG. 5 and reservoir 123 from FIG. 6. Sparger 635 performs the same function as sparger 227 from FIG. 5 and sparger 123 from FIG. 6.

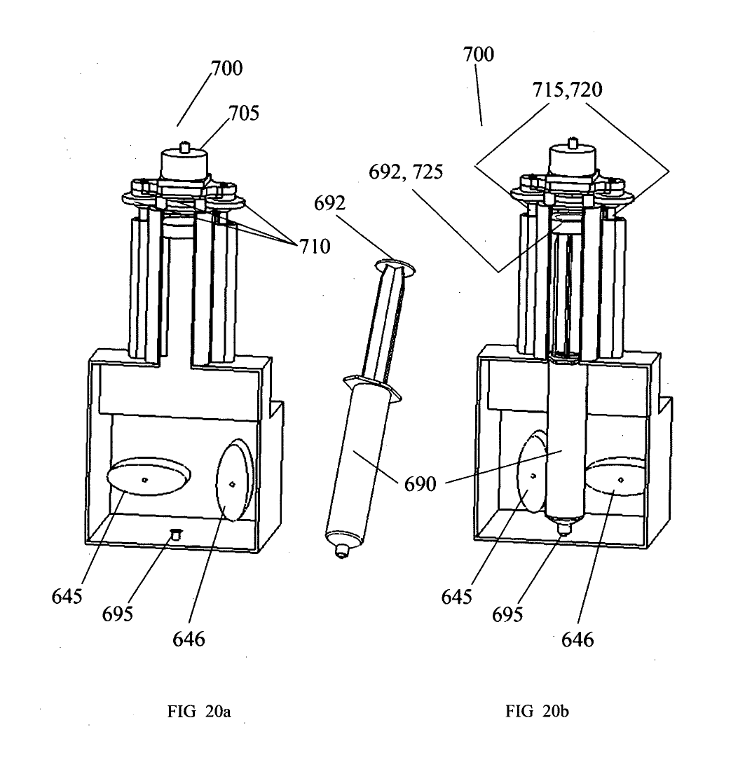

FIGS. 20(a), 20(b) and 20(c) show an embodiment of a syringe pump 700 for use with the humidification/decontamination system of the present invention. FIG. 20(a) shows decontamination valve 645 closed and humidification valve 646 open. FIG. 20(c) shows decontamination valve 645 open and humidification valve 646 closed with sterilant syringe 690 attached to luer lock connector 695. Gears and/or other linkages (not shown) could optionally be used to ensure that valves 645 and 646 are never both open at the same time.

Syringe Pump 700 is comprised of a rotary stepper motor 705 and three gears 710 that rotate lead screws 715 and 720 that operate on follower 725 causing follower 725 to push plunger 692 of syringe 690 down during a decontamination cycle propelling liquid sterilant through conduit 154 to atomization nozzle 136 in tee 135. The atomized liquid sterilant merges with the hot, humidity controlled air stream that is flowing down conduit 134, through tee 135 and down conduit 137. When the air exits conduit 137 and passes through tee 660 on its way to filter 670 and conduit 680 the liquid sterilant mist will be vaporized so that only air and sterilant vapors are carried into the incubator chamber 5.

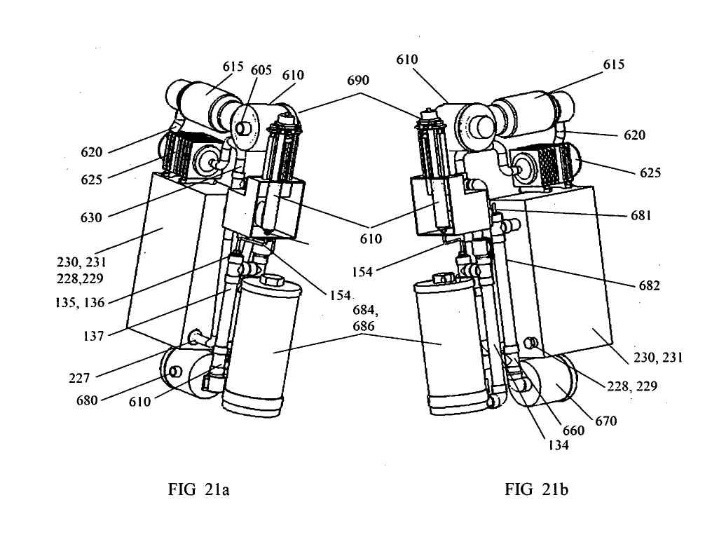

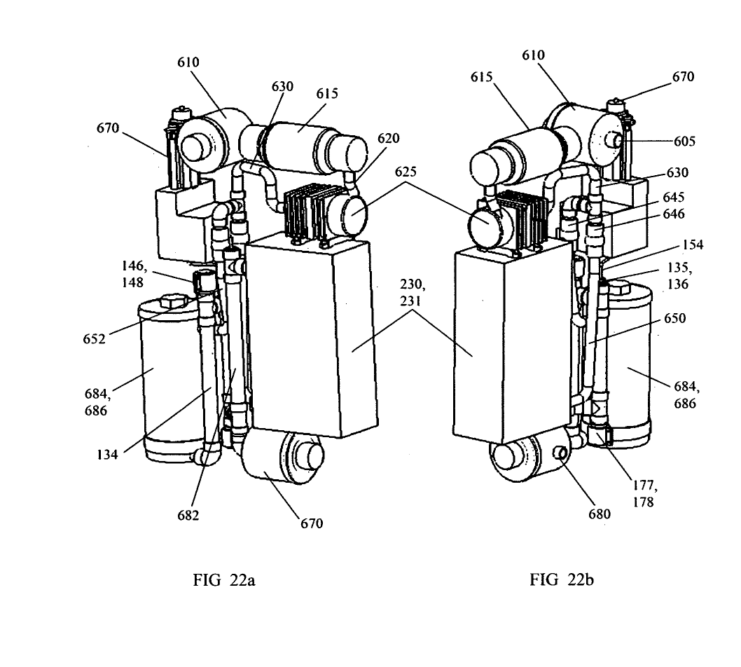

Refer to FIGS. 21(a), 21(b), 22(a) and 22(b) for a system that is capable of performing the decontamination cycle shown in FIG. 15. The water vapor in the recirculating air stream is removed by a desiccant air dryer so that the maximum amount of H₂O₂ vapor can be atomized, vaporized and continuously passed into, through and out of the incubator chamber 5.

Air is withdrawn from the incubator chamber through conduit 605, HEPA filter 610, catalytic converter 615 and conduit 620 by air pump 625. The air is discharged from air pump 625 through conduit 630 which connects to valves 645 and 646. When valve 645 is open, valve 646 is closed. When valve 646 is open valve 645 is closed. Thus, the air exiting conduit 630 can go through either valve 645 or valve 646.

During humidification, valve 646 is open so the air passed through conduit 650 and sparger 227 and into reservoir 230 filled with water 231 that is heated by heater 228. Temperature sensor 229 provides feedback to the microprocessor that is used to control heater 228. The air exits sparger 227 as micro-bubbles that float to the surface and exit into the air space above water 231 at near 100% relative humidity based upon the temperature of water 231. The humid air stream exits reservoir 230 through conduit 682 that contains optional temperature and/or humidity sensor 681. The humid air passes through conduit 682, through tee 660, HEPA filter 670 and port 680 as it is returned to the incubator chamber 5.

During sterilization/decontamination, valve 645 is open so the air stream exits conduit 630 and passes into and through conduit 652 on its way to air dryer 684 filled with desiccant 686. Desiccant 686 is contained in a porous sack, or bag, that conforms to the inside geometry of air dryer 684. This sack, or bag, can be made of a cloth and may have a “draw string” or access on one end. The desiccant 686 can be removed from air dryer 684 at the completion of the decontamination process. The desiccant bag can be replaced prior to running each contamination cycle. The replacement bag can contain “new” or regenerated desiccant.

The air stream exits air dryer 684 through conduit 134. As the air passes through conduit 134 it is warmed by heater 146 whose temperature is monitored by temperature sensor 148. The hot air stream exiting conduit 135 merges with nebulized liquid sterilant from liquid delivery line 154 in tee 135. The mixture of nebuilzed liquid sterilant and hot air exits tee 135 and passed into heated conduit 137 and into and through tee 660, HEPA filter 670 and conduit 680 as it is returned to the incubator chamber 5.

1. Laboratory equipment having an enclosed chamber for storage of samples at a predetermined temperature and humidity level and in a sterile condition, comprising:—
a humidification system which automatically maintains the temperature and humidity levels within the chamber at preselected levels without user intervention for an indefinite period of time once the system has been installed, the required utilities connected and the desired settings including temperature and humidity level programmed; and
a decontamination system which automatically decontaminates the chamber without user intervention other than selecting the desired decontamination cycle, installing a sterilant cartridge and initializing a decontamination cycle.

2. The laboratory equipment of claim 1, wherein said humidification system comprises a depth of water maintained at a controlled temperature, and wherein the humidification system draws air from said enclosed chamber, passes it in the form of small bubbles through said depth of water to produce the desired humidity level and the humidified air is returned to said enclosed chamber.

3. The laboratory equipment of claim 2 wherein the depth of said depth of water is maintained by a float valve connected to a deionized water supply line.

4. The laboratory equipment of claim 1, wherein said decontamination system comprises a vaporous sterilant comprised of at least hydrogen peroxide vapor and water vapor.

5. The laboratory equipment of claim 4, wherein said decontamination system further comprises a depth of water maintained at a controlled temperature, and wherein said decontamination system draws air from said enclosed chamber, passes it in the form of small bubbles through said depth of water to pre-condition the air to the desired humidity level.

6. The laboratory equipment of claim 4, wherein said decontamination system further comprises a depth of water, and wherein said decontamination system discharges degraded vaporous sterilant and air in the form of small bubbles into said depth of water, where said sterilant vapors are captured and condensed while said air is passed.

7. The laboratory equipment of claim 4, wherein said decontamination system pre-conditioned air is returned to the enclosed chamber with a pump for a time period sufficient to precondition the air within said enclosed chamber.

8. The laboratory equipment of claim 5, wherein said combination of pre-conditioned air at a controlled flow rate, liquid sterilant at a controlled nebulization/vaporization rate and air in said enclosed chamber at a controlled temperature is sufficient to maintain the desired sterilant vapor concentration and desired percent saturation during a sterilization exposure phase.

9. The laboratory equipment of claim 5 wherein said depth of water is chilled.

10. (canceled)

11. (canceled)

12. (canceled)

13. (canceled)

14. The laboratory equipment of claim 5, further comprising a pump for supplying vaporous sterilant.

15. An integrated automatic humidification and decontamination system for the laboratory equipment of claim 1, comprising a catalytic converter.

16. An integrated automatic humidification and decontamination system for the laboratory equipment of claim 1, comprising a syringe pump to pump liquid sterilant vapor during decontamination.

17. An integrated automatic humidification and decontamination system for the laboratory equipment of claim 1, comprising an air pump to supply liquid sterilant vapor during decontamination.

18. An integrated automatic humidification and decontamination system for the laboratory equipment of claim 1, comprising an air dryer containing desiccant to pre-condition air during the decontamination stage.

19. (canceled)

20. (canceled)

21. (canceled)

22. (canceled)

23. (canceled)

24. A method to carry out decontamination using an integrated automatic humidification and decontamination system for laboratory equipment, comprising the following steps:—
drawing air from an enclosed chamber, passing the air in the form of small bubbles through a depth of water that is maintained at a controlled temperature to provide a preconditioned air stream for the decontamination step,
nebulizing a liquid sterilant into said preconditioned air stream,
vaporizing said nebulized liquid sterilant to provide sterilant vapor laden air,
returning said sterilant vapor laden air to said enclosed chamber,
maintaining a desired sterilant vapor concentration and desired percent saturation during the sterilization exposure step by keeping the pre-conditioned air in said pre-conditioned air stream at a controlled flow rate, whereby the liquid sterilant at a controlled vaporization rate and the air in said enclosed chamber at a controlled temperature discharges degraded vaporous sterilant and air in the form of small bubbles into a depth of water that condenses and captures the sterilant vapors and passes the air, upon completion of the decontamination step,
returning the pre-conditioned air to said enclosed chamber for a time period sufficient to precondition the air within said enclosed chamber, and upon completion of the decontamination step,
the humidification system draws air from said enclosed chamber, passes it in the form of small bubbles through a depth of water that is maintained at a controlled temperature to produce the desired humidity level to continue operation of the laboratory equipment after the decontamination step.

25. An integrated humidification and decontamination module that can be retrofitted onto laboratory equipment having an enclosed chamber for storage of samples at a predetermined temperature, humidity and CO₂ levels and in a sterile condition, said integrated humidification and decontamination system comprising:—
a humidification system which automatically maintains the temperature, and humidity levels within said enclosed chamber at their preselected levels without user intervention for an indefinite period of time once the system has been installed, the required utilities connected and the desired settings including temperature and humidity level programmed; and
a decontamination system which automatically decontaminates said enclosed chamber without user intervention other than selecting the desired decontamination cycle, installing the sterilant cartridge and initializing a decontamination cycle.
